SEQUENCE LISTING (45 sequences)

SEQ ID NO: 1 - DNA, 810 bp, Homo sapiens
  CDS (1)..(810)
atgcatattttaaaagggtctcccaatgtgattccacgggctcacgggcagaagaacacgcgaagagacggaactggcctctatcctatgcgaggtccctttaagaacctcgccctgttgcccttctccctcccgctcctgggcggaggcggaagcggaagtggcgagaaagtgtcggtctccaagatggcggccgcctggccgtctggtccgtctgctccggaggccgtgacggccagactcgttggtgtcctgtggttcgtctcagtcactacaggaccctggggggctgttgccacctccgccgggggcgaggagtcgcttaagtgcgaggacctcaaagtgggacaatatatttgtaaagatccaaaaataaatgacgctacgcaagaaccagttaactgtacaaactacacagctcatgtttcctgttttccagcacccaacataacttgtaaggattccagtggcaatgaaacacattttactgggaacgaagttggttttttcaagcccatatcttgccgaaatgtaaatggctattcctacaaagtggcagtcgcattgtctctttttcttggatggttgggagcagatcgattttaccttggataccctgctttgggtttgttaaagttttgcactgtagggttttgtggaattgggagcctaattgatttcattcttatttcaatgcagattgttggaccttcagatggaagtagttacattatagattactatggaaccagacttacaagactgagtattactaatgaaacatttagaaaaacgcaattatatccataa

SEQ ID NO: 2 - protein, 269 aa, Homo sapiens
MHILKGSPNVIPRAHGQKNTRRDGTGLYPMRGPFKNLALLPFSLPLLGGGGSGSGEKVSVSKMAAAWPSGPSAPEAVTARLVGVLWFVSVTTGPWGAVATSAGGEESLKCEDLKVGQYICKDPKINDATQEPVNCTNYTAHVSCFPAPNITCKDSSGNETHFTGNEVGFFKPISCRNVNGYSYKVAVALSLFLGWLGADRFYLGYPALGLLKFCTVGFCGIGSLIDFILISMQIVGPSDGSSYIIDYYGTRLTRLSITNETFRKTQLYP

SEQ ID NO: 3 - DNA, 962 bp, Homo sapiens
  CDS (14)..(655)
tgtgcccgggaagatggtgctaggtggttgcccggttagttacttacttctgtgcggccaggcggctttgctgctggggaatttacttctgctgcattgtgtgtctcggagccactcgcaaaatgcgaccgctgagcctgagctcacatccgctggcgccgcccagccggagggccccgggggtgctgcgagctgggaatatggcgacccccactctccggtcatcctctgctcttacctacctgatgaatttatagaatgtgaagacccagtggatcatgttggaaatgcaactgcatcccaggaacttggttatggttgtctcaagttcggcggtcaggcctacagcgacgtggaacacacttcagtccagtgccatgccttagatggaattgagtgtgccagtcctaggacctttctacgagaaaataaaccttgtataaagtataccggacactacttcataaccactttactctactccttcttcctgggatgttttggtgtggatcgattctgtttgggacacactggcactgcagtagggaagctgttgacgcttggaggacttgggatttggtggtttgttgaccttattttgctaattactggagggctgatgccaagtgatggcagcaactggtgcactgtttactaaaaagagctgccatcatggcccagggaggcgggtgaaagctccgtcttctgaattcatctctacaggctcaaaactcctctttgatatcagacctgatgttattttccttcttttggagggcatttgtttggttaagaaggcttctttggactttggaatttcaacccagattttaccttgcagacggaatgacaagcaaaaagtgttgtggggaatcaaatttgttcctttcctcatgcacaaaacataaaggatagtggcgagtttacaagctgtggatgggtttccatagtcttcctttctg

SEQ ID NO: 4 - protein, 214 aa, Homo sapiens
MVLGGCPVSYLLLCGQAALLLGNLLLLHCVSRSHSQNATAEPELTSAGAAQPEGPGGAASWEYGDPHSPVILCSYLPDEFIECEDPVDHVGNATASQELGYGCLKFGGQAYSDVEHTSVQCHALDGIECASPRTFLRENKPCIKYTGHYFITTLLYSFFLGCFGVDRFCLGHTGTAVGKLLTLGGLGIWWFVDLILLITGGLMPSDGSNWCTVY

SEQ ID NO: 5 - DNA, 746 bp, Homo sapiens
  CDS (6)..(668)
gcaagatggcgggaggggtgcgcccgctgaggggcctccgcgccttgtgtcgcgtgctgctcttcctctcgcagttctgcattctgtcgggcggtgaaagtactgaaatcccaccttatgtgatgaagtgtccgagcaatggtttgtgtagcaggcttcctgcagactgtatagactgcacaacaaatttctcctgtacctatgggaagcctgtcacttttgactgtgcagtgaaaccatctgttacctgtgttgatcaagacttcaaatcccaaaagaacttcatcattaacatgacttgcagattttgctggcagcttcctgaaacagattacgagtgtaccaactccaccagctgcatgacggtgtcctgtcctcggcagcgctaccctgccaactgcacggtgcgggaccacgtccactgcttgggtaaccgtacttttcccaaaatgctatattgcaattggactggaggctataagtggtctacggctctggctctaagcatcaccctcggtgggtttggagcagaccgtttctacctgggccagtggcgggaaggcctcggcaagctcttcagcttcggtggcctgggaatatggacgctgatagacgtcctgctcattggagttggctatgttggaccagcagatggctctttgtacatttagctgtggtgtgtgcttcagaaaggagcagggcttagaaaaagcccttttgtccgtagagttgatgtggtgtgagtg

SEQ ID NO: 6 - protein, 221 aa, Homo sapiens
MAGGVRPLRGLRALCRVLLFLSQFCILSGGESTEIPPYVMKCPSNGLCSRLPADCIDCTTNFSCTYGKPVTFDCAVKPSVTCVDQDFKSQKNFIINMTCRFCWQLPETDYECTNSTSCMTVSCPRQRYPANCTVRDHVHCLGNRTFPKMLYCNWTGGYKWSTALALSITLGGFGADRFYLGQWREGLGKLFSFGGLGIWTLIDVLLIGVGYVGPADGSLYI

SEQ ID NO: 7 - DNA, 22 bp, Homo sapiens
tgtgcccgggaagatggtgcta

SEQ ID NO: 8 - DNA, 22 bp, Homo sapiens
cagaaaggaagactatggaaac

SEQ ID NO: 9 - DNA, 24 bp, Homo sapiens
cactcacaccacatcaactctacg

SEQ ID NO: 10 - DNA, 40 bp, Homo sapiens
taatacgactcactatagggttagaagaaacagatttgag

SEQ ID NO: 11 - DNA, 40 bp, Homo sapiens
attaaccctcactaaagggacaagtggcaacttgcctttg

SEQ ID NO: 12 - DNA, 40 bp, Homo sapiens
taatacgactcactatagggaagagctgccatcatggccc

SEQ ID NO: 13 - DNA, 40 bp, Homo sapiens
attaaccctcactaaagggaaaaggaagactatggaaacc

SEQ ID NO: 14 - DNA, 40 bp, Homo sapiens
taatacgactcactatagggcctgggccagtggcgggaag

SEQ ID NO: 15 - DNA, 40 bp, Homo sapiens
attaaccctcactaaagggacactcacaccacatcaactc

SEQ ID NO: 16 - DNA, 15 bp, Homo sapiens
cccccatgccatcct

SEQ ID NO: 17 - DNA, 24 bp, Homo sapiens
gactcgtcatactcctgcttgctg

SEQ ID NO: 18 - DNA, 24 bp, Homo sapiens
agatcgattttaccttggataccc

SEQ ID NO: 19 - DNA, 24 bp, Homo sapiens
gagacagaagcccgagaaacacta

SEQ ID NO: 20 - DNA, 24 bp, Homo sapiens
gaattcatctctacaggctcaaaa

SEQ ID NO: 21 - DNA, 24 bp, Homo sapiens
cacggccatttctatttctgctga

SEQ ID NO: 22 - DNA, 24 bp, Homo sapiens
gcagcttcctgaaacagattacga

SEQ ID NO: 23 - DNA, 24 bp, Homo sapiens
caccacatcaactctacggacaaa

SEQ ID NO: 24 - DNA, 30 bp, Homo sapiens
ccttccatggaagtggcagtcgcattgtct

SEQ ID NO: 25 - DNA, 32 bp, Homo sapiens
aacactcgagtcaaaaccctacagtgcaaaac

SEQ ID NO: 26 - DNA, 28 bp, Homo sapiens
ccatggccactttactctactccttctt

SEQ ID NO: 27 - DNA, 29 bp, Homo sapiens
ctcgagtcaaatcccaagtcctccaagcg

SEQ ID NO: 28 - DNA, 27 bp, Homo sapiens
ccatggctctggctctaagcatcaccc

SEQ ID NO: 29 - DNA, 29 bp, Homo sapiens
ctcgagtcatattcccaggccaccgaagc

SEQ ID NO: 30 - DNA, 22 bp, Homo sapiens
gtggatccactgcttcgaggat

SEQ ID NO: 31 - DNA, 28 bp, Homo sapiens
gtcgacggttgctatacaggacaagagg

SEQ ID NO: 32 - DNA, 22 bp, Homo sapiens
gtggatccagtgcttcaatgat

SEQ ID NO: 33 - DNA, 28 bp, Homo sapiens
gtcgactaaatttgggcgttcccttctt

SEQ ID NO: 34 - DNA, 22 bp, Homo sapiens
gtggatccactgctttgagggt

SEQ ID NO: 35 - DNA, 28 bp, Homo sapiens
gtcgacggtcttcttgcccccatcttcc

SEQ ID NO: 36 - DNA, 33 bp, Homo sapiens
tggtgaattcgaaagtgtcggtctccaagatgg

SEQ ID NO: 37 - DNA, 33 bp, Homo sapiens
cttcgtcgacttatggatataattgcgtttttc

SEQ ID NO: 38 - DNA, 33 bp, Homo sapiens
ttccgaattcaagatggtgctaggtggttgccc

SEQ ID NO: 39 - DNA, 33 bp, Homo sapiens
ttccctcgagttagtaaacagtgcaccagttgc

SEQ ID NO: 40 - DNA, 33 bp, Homo sapiens
ttttgaattcgcaagatggcgggaggggtgcgc

SEQ ID NO: 41 - DNA, 33 bp, Homo sapiens
ttggctcgagctaaatgtacaaagagccatctg

SEQ ID NO: 42 - DNA, 34 bp, Homo sapiens
ggttgggagcagatgcattttaccttggataccc

SEQ ID NO: 43 - DNA, 34 bp, Homo sapiens
ggttgggagcagatgaattttaccttggataccc

SEQ ID NO: 44 - DNA, 41 bp, Homo sapiens
ctgggatgttttggtgtggatgaattctgtttgggacacac

SEQ ID NO: 45 - DNA, 39 bp, Homo sapiens
ggtgggtttggagcagacgaattctacctgggccagtgg

(19) United States
(12) Patent Application Publication
(10) Pub. No.: US 2003/0096356 A1
(43) Pub. Date: May 22, 2003

(54) Novel G-protein-coupled receptor like proteins and polynucleotides encoded by them, and methods of using same

(75) Inventors: Bradley A. Ozenberger, Newtown, PA (US); Eileen M. Kajkowski, Ringoes, NJ (US); Ching-Hsiung Frederick Lo, Pennington, NJ (US); Heidi Sofia, Walla Walla, WA (US)

Correspondence Address: Gavin T. Bogle, Patent Law Department, 5 Giralda Farms, Madison, NJ, 07940, US

(73) Assignee: Wyeth, Madison, NJ

(21) Appl. No.: 10/199,881

(22) Filed: Jul. 18, 2002

Related U.S. Application Data
(63) Continuation of application No. 09/833,503, filed on Apr. 12, 2001.
(60) Provisional application No. 60/104,104, filed on Oct. 13, 1998.

(30) Foreign Application Priority Data
Oct. 13, 1999 (WO) PCT/US99/21621

(51) Int. Cl. C07K 14/705; C07H 21/04; C12P 21/02; C12N 5/06

(52) U.S. Cl. 435/69.1; 435/320.1; 435/325; 530/350; 536/23.5

(57) ABSTRACT

Novel proteins which contain a structural module conserved in the G protein coupled receptor superfamily, polynucleotides which encode these proteins, and methods for producing these proteins are provided. Diagnostic, therapeutic, and screening methods employing the polynucleotides and polypeptides of the present invention are also provided.

### RELATED APPLICATIONS

This application is a continuation of copending application Ser. No. 09/833,503 filed on Apr. 12, 2001, the entire disclosure of which is hereby incorporated by reference. This application claims priority from PCT Application No. PCT/US99/21621, filed Oct. 13, 1999 and U.S. Provisional Application No. 60/104,104 filed Oct. 13, 1998.

### FIELD OF THE INVENTION

The present invention relates to a novel polynucleotides and proteins encoded by such polynucleotides, along with therapeutic, diagnostic, and research utilities for these polynucleotides and proteins. In particular, the invention relates to polynucleotides and proteins encoded by such polynucleotides which comprise a structural module that is conserved in the G-protein-coupled receptor (“GPCR”) superfamily and that can modulate apoptosis signaling pathways.

### BACKGROUND OF THE INVENTION

The actions of many extracellular signals are mediated by receptors with seven transmembrane domains (G protein coupled receptors, “GPCR”) and heterotrimeric guanine nucleotide binding regulatory proteins G proteins. G proteins are important to regulatory mechanisms operating in all human cells. Impairment of their function can perturb the cell's response to hormonal signals and adversely affect many intracellular metabolic pathways, thus contributing to the development and maintenance of a wide variety of disease states.

When functioning normally, G proteins act as an integral part of the signal transducing mechanism by which extracellular hormones and neurotransmitters convey their signals through the plasma membrane of the cell and thus elicit appropriate intracellular responses.

In its simplest terms, the signal transducing mechanism can be said to comprise three distinct components: (a) a receptor protein with an extracellular binding site specific for a given agonist, such as the beta-adrenergic receptor; (b) effector protein (an enzyme) that, when activated, catalyzes the formation or facilitates the transport of an intracellular second messenger; an example is adenylate cyclase which produces cyclic AMP (cAMP); and (c) a third protein which functions as a communicator between the receptor protein and the membrane bound effector protein. G proteins fulfill this vital role as communicator in the generation of intracellular responses to extracellular hormones and agonists (i.e., signal transduction).

G proteins are composed of three polypeptide subunits, namely G alpha (G_), G beta (G_) and G gamma (G_) (3). The conformation of each subunit and their degree of association change during the signal transducing mechanism. These changes are associated with the hydrolysis of GTP (GTPase activity) to form GDP and Pᵢ. The binding sites for GTP, GDP and the GTPase activity reside in the alpha subunit.

These integral membrane proteins which modulate the activity of heterotrimeric G proteins have a common topology, transversing the membrane seven times, as described above. Due to their important functions, and the immense size of the gene family (estimated to contain >10,000 members in the human genome), GPCRs have been intensively researched.

Due to their importance in human pharmacology, G protein and GPCRs continue to be exhaustively studied.

### SUMMARY OF THE INVENTION

A first aspect of this invention is the discovery of a novel gene (and protein) family containing a segment related to the GPCR superfamily. This new gene family-presently contains three members denoted BBP1, BBP2 and BBP3. The proteins are predicted to transverse the membrane twice via a structural module that is equivalent to transmembrane domains 3 and 4 of 7-transmembrane domain GPCRs. The remaining sequences of the novel BBP proteins share no significant homology with other known proteins.

In a preferred embodiment, the novel BBPs contain the protein motif “DRF”, highly conserved in all members of the GPCR family which, in GPCRs, acts as the biochemical activator of heterotrimeric G proteins. In another aspect of the invention, it was demonstrated that the BBP proteins physically interact with G-alpha proteins in yeast 2 hybrid (Y2H) assays, suggesting that the module may serve the same function in BBPs as it does in GPCRs; namely, to regulate the activity of G protein signaling pathways.

In a further aspect of the present invention, the distribution of the novel BBP mRNAs is examined in human and tumorigenic tissues. Investigations of BBP gene expression in tumors and cancer cell lines demonstrated that these genes are overexpressed in some tumors and their expression can be observed in many cell lines.

In yet another embodiment of the invention, a cell culture system for recombinant expression demonstrated that all three BBPs suppress apoptosis induction as measured by the incidence of condensed nuclei, and that substitution of the arginine in the ‘DRF’ motif abrogates protection. This evidence suggests that BBPs act as modulators of cell survival signals, and that integration with such pathways may occur through heterotrimeric G proteins.

### BRIEF DESCRIPTION OF THE DRAWINGS

FIG. 1. BBP protein alignment. The BBP proteins were aligned using the ClustalW algorithm (Thompson et al., 1994). The BBP1 [SEQ ID NO:2] protein shown initiates at the third potential translation start site. Identical and similar amino acids are shaded and boxed. The predicted tm domains are indicated by lines labeled tm1 and tm2. The stars indicate specific residues which are conserved in at least 85% of all known GPCRs and also contained within all three BBPs at homologous locations (GPCR tm3=BBP tm1; GPCR tm4=BBP tm2). 96% of GPCRs contain a W near the center of tm4; this residue is conserved in BBP2 [SEQ ID NO:4] and BBP3 [SEQ ID NO:6] but absent in BBP1.

FIG. 2. Expression of BBP1 mRNA in human tissues. Nylon membranes blotted with 2 μg size fractionated poly-A RNA isolated from the indicated tissues were obtained from Clontech Laboratories, Inc. These were hybridized with a radiolabeled BBP1 cDNA probe as described. A predominant band corresponding to 1.25 kb (determined from molecular weight markers, not shown) was observed in all lanes. Higher molecular weight bands likely correspond to heteronuclear RNA; the BBP1 gene contains several introns (data not shown). Blots were stripped and reprobed with β-actin as a loading and RNA integrity control; all lanes exhibited equivalent signal (data not shown).

FIG. 3. Expression of BBP2 mRNA in human tissues. Expression of BBP2 was determined as described in the legend to FIG. 2. The BBP2 transcript is approximately 1.35 kb in length.

FIG. 4. Expression of BBP3 mRNA in human tissues. Expression of BBP3 was determined as described in the legend to FIG. 2. The BBP3 transcript is approximately 1.40 kb in length.

FIG. 5. Expression of BBP mRNAs in human tissues. A nylon membrane spotted with mRNAs isolated from 50 human tissues was obtained from Clontech Laboratories. It was sequentially stripped and hybridized with radiolabeled probes derived from each BBP cDNA, and ubiquitin as a control. The autoradiograms shown are A. BBP1, B. BBP2, C. BBP3, D. ubiquitin. The tissue samples are as follows: row 1, whole brain, amygdala, caudate nucleus, cerebellum, cerebral cortex, frontal lobe, hippocampus, medulla oblongata; row 2, occipital lobe, putamen, substantia nigra, temporal lobe, thalamus, subthalamic nucleus, spinal cord; row 3, heart, aorta, skeletal muscle, colon, bladder, uterus, prostate, stomach; row 4, testis, ovary, pancreas, pituitary gland, adrenal gland, thyroid gland, salivary gland, mammary gland; row 5, kidney, liver, small intestine, spleen, thymus, peripheral leukocyte, lymph node, bone marrow; row 6, appendix, lung, trachea, placenta; row 7, fetal brain, fetal heart, fetal kidney, fetal liver, fetal spleen, fetal thymus, fetal lung.

FIG. 6. Expression of BBP1 in nonhuman primate brain. Autoradiograms of coronal sections of cynomolgus monkey forebrain taken at rostral (A), mid (B), and caudal levels (C and D), processed to visualize the distribution of BBP1 mRNA by in situ hybridization histochemistry as described in Materials and Methods. Darker areas of the image correspond to areas of higher expression of BBP1 mRNA.

FIG. 7. Expression of BBP2 in nonhuman primate brain. Autoradiograms of coronal sections of cynomolgus monkey forebrain as described in the legend to FIG. 6. Darker areas of the image correspond to areas of higher expression of BBP2 mRNA.

FIG. 8. Expression of BBP3 in nonhuman primate brain. Autoradiograms of coronal sections of cynomolgus monkey forebrain as described in the legend to FIG. 6. Darker areas of the image correspond to areas of higher expression of BBP3 mRNA.

FIG. 9. Comparison of BBP1 expression in tumors and corresponding normal tissue samples. A nylon membrane blotted with 20 μg total RNA isolated from the indicated human sources was obtained from Invitrogen Corp. It was hybridized with a radiolabeled BBP1 probe as described. The same blot was stripped and reprobed with a β-actin probe as a loading and RNA integrity control.

FIG. 10. Examination of BBP gene expression in tumors and corresponding normal tissue samples. A nylon membrane blotted with 20 μg total RNA isolated from the indicated human sources was obtained from Invitrogen Corp. It was sequentially stripped and hybridized with radiolabeled probes as indicated by the labels. Ubiquitin was used as a control.

FIG. 11. Examination of BBP gene expression in female tissue tumors and corresponding normal samples. Methods are as described in the legend to FIG. 10.

FIG. 12. Examination of BBP gene expression in cancer cell lines. Methods are as described in the legend to FIG. 5 except ubiquitin was used as a control. The cell lines are HL-60, promyelocytic leukemia; HeLa S3, carcinoma; K-562, chronic myelogenous leukemia; MOLT-4, lymphoblastic leukemia; Raji, Burkitt's lymphoma; SW480, colorectal adenocarcinoma; A549, lung carcinoma; G361, melanoma.

FIG. 13. Bioassay for BBP1 interactions with G_proteins. The intracellular domain of BBP1 was expressed as a Gal4 DNA-binding domain fusion protein with rat Gαs, Gαo, or Gαi2 Gal4 activation domain fusion proteins and Y2H growth responses were compared to cells lacking a G protein component (vector) on assay medium as described in Materials and Methods. Dual columns represent independently derived isolates of the same strain. The number of cells applied to the medium decreases by 10-fold in each row.

FIG. 14. Bioassay for BBP2 interactions with Ga proteins. The intracellular domain of BPP2 was expressed as a Gal4 DNA-binding domain fusion protein with rat Gαs, Gαo, or Gαi2 Gal4 activation domain fusion proteins and Y2H growth responses were compared to cells lacking a G protein component (vector), as described in the legend to FIG. 13.

FIG. 15. Bioassay for BBP3 interactions with G_proteins. The intracellular domain of BBP3 was expressed as a Gal4 DNA-binding domain fusion protein with rat Gαs, Gαo, or Gαi2 Gal4 activation domain fusion proteins and Y2H growth responses were compared to cells lacking a G protein component (vector), as described in the legend to FIG. 13.

FIG. 16. BBP1 suppresses staurosporine-induced nuclear condensation (apoptosis). Nt2 stem cells were transfected with pEGFP alone (columns 1 and 4), pEGFP plus p5HT1a (columns 2 and 5), or pEGFP plus pOZ363 (BBP1; columns 3 and 6). Samples were untreated (columns 1-3) or treated with 100 nM staurosporine for 3 hrs (columns 4-6). Values represent the mean percentage of condensed nuclei among transfectants (EGFP+) of duplicate samples. Error bars indicate the standard error of the mean.

FIG. 17. Substitutions of the arginine in the ‘DRF’ motif in BBP1 attenuate the suppression of apoptosis. The BBP1-R138A and BBP1-R138E expression plasmids are identical to BBP1-wt except for the codon at position 138. Results are represented as described in the legend to FIG. 16 except data were drawn from triplicate samples. Values with the same superscript letter are significantly different (P<0.05) as determined by Yates modified chi-square test of probability. The staurosporine treated BBP1-wt samples (column 6) were significantly different from control or R138 substitution samples with P<0.005.

FIG. 18. All three BBP protein subtypes suppress staurosporine-induced nuclear condensation. Nt2 stem cells were transfected with pEGFP alone or pEGFP plus a plasmid expressing the indicated BBP protein as described in the text. Results are represented as described in the legend to FIG. 16.

FIG. 19. The R to E substitution in the BBP2 ‘DRF’ motif substantially reduces suppression of staurosporine-induced nuclear condensation. Results are represented as described in the legend to FIG. 15 except nontreated controls are not shown.

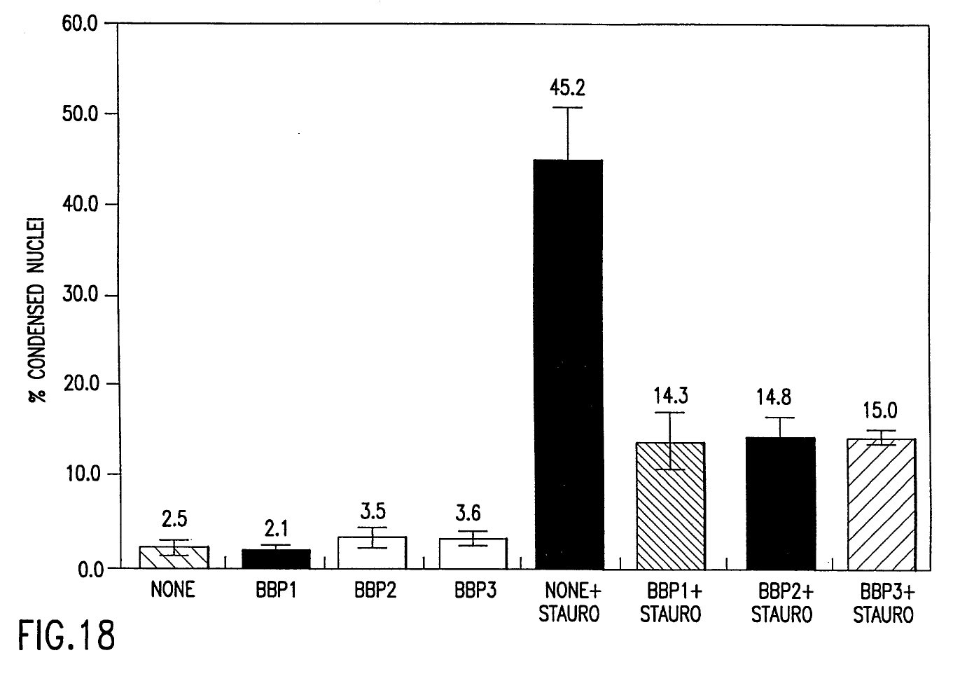

FIG. 20. The R to E substitution in the BBP3 ‘DRF’ motif substantially reduces suppression of staurosporine-induced nuclear condensation. Results are represented as described in the legend to FIG. 15 except nontreated controls are not shown.

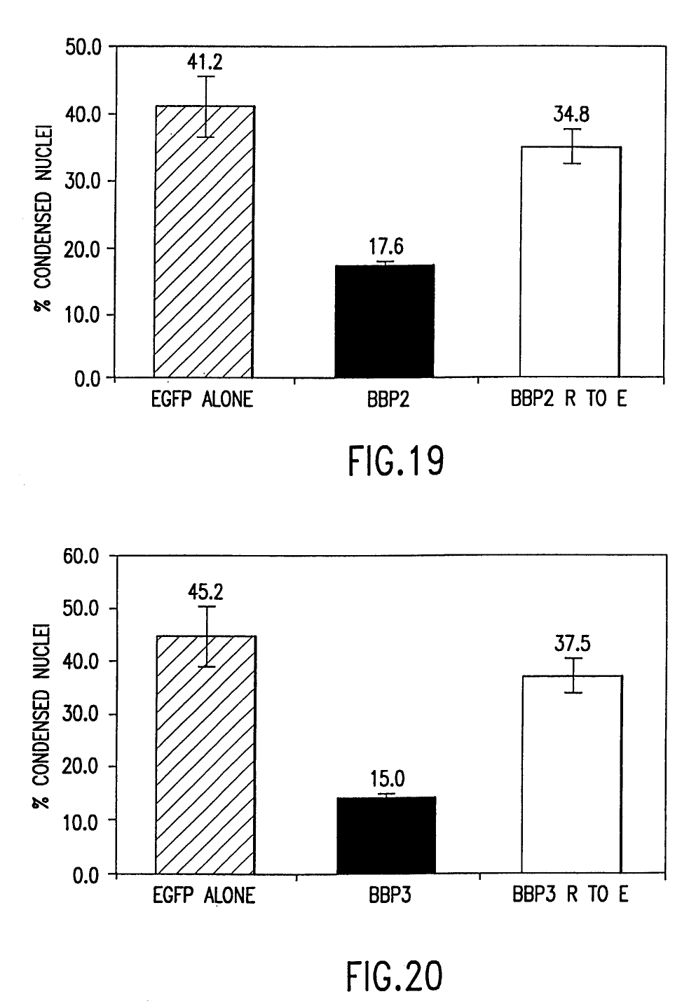

### DETAILED DESCRIPTION OF INVENTION

### Definitions

A “chemical” is defined to include any drug, compound or molecule.

A G-protein-coupled receptor or “GPCR” is defined to be any transmembrane protein that when activated by a chemical in turn activates a heterotrimeric guanine nucleotide-binding protein (G-protein).

“Apoptosis” is defined herein to be programmed cell death, in particular suppression of nuclear condensation induced by staurosporine.

Identification of BBP1. β-amyloid peptide (BAP) is the principal constituent of neuritic senile plaques and is a central focus of Alzheimer's disease (AD) research. Numerous findings indicate that BAP is a causative factor in the neuron death and consequent diminution of cognitive abilities observed in AD sufferers (reviewed by Selkoe, 1997). To better understand the mechanism by which β-amyloid peptide induces neuronal cell death, a yeast 2-hybrid (Y2H) genetic screen was developed to identify proteins which interact with human BAP42. The screen, described elsewhere (patent application co-owned and co-pending Ser. No. 09/060,609), identified a cDNA encoding a novel BAP binding protein (BBP1).

Identification of additional BBP DNA sequences. The Genbank database was probed for BBP1-like DNA and protein sequences using the basic local alignment search tool (BLAST; Altschul et al., 1990). Two Caenorhabditis elegans and one Drosophila melanogaster genomic sequence and a large number of human, mouse and other mammalian expressed sequence tags (ESTs) were identified. However, no complete cDNA sequences were available nor were any functional data attributed to the Genbank items. [The C. elegans BBP1-related sequences in Genbank are included within cDNAs assembled erroneously from the genomic DNA sequence (data not shown)]. All BBP ESTs were extracted from the database and aligned, revealing three distinct sets of DNAs and, therefore, three BBP gene and protein subtypes. All three BBP subtypes are represented in both human and mouse data sets. Exhaustive analysis of the Genbank database failed to identify additional subtypes.

The Coding Sequence for BBPs

In accordance with the present invention, nucleotide sequences which encode BBPs, fragments, fusion proteins or functional equivalents thereof, may be used to generate recombinant DNA molecules that direct the expression of BBPs, or functionally active peptides, in appropriate host cells. Alternatively, nucleotide sequences which hybridize to portions of BBP sequences may be used in nucleic acid hybridization assays, Southern and Northern blot assays, etc.

The invention also includes polynucleotides with sequences complementary to those of the polynucleotides disclosed herein.

The present invention also includes polynucleotides capable of hybridizing under reduced stringency conditions, more preferably stringent conditions, and most preferably highly stringent conditions, to polynucleotides described herein. Examples of stringency conditions are shown in the table below: highly stringent conditions are those that are at least as stringent as, for example, conditions A-F; stringent conditions are at least as stringent as, for example, conditions G-L; and reduced stringency conditions are at least as stringent as, for example, conditions M-R.

Stringency Conditions

| | Poly- | Hybrid | Hybridization | Wash |
|---|---|---|---|---|
| Stringency | nucleotide | Length | Temperature and | Temperature |
| Condition | Hybrid | (bp)^{I} | BufferH | and BufferH |
| A | DNA:DNA | >50 | 65EC; 1xSSC -or- | 65EC; 0.3xSSC |
| | | | 42EC; 1xSSC, 50% | |
| | | | formamide | |
| B | DNA:DNA | <50 | T_{B}*; 1xSSC | T_{B}*; 1xSSC |
| C | DNA:RNA | >50 | 67EC; 1xSSC -or- | 67EC; 0.3xSSC |
| | | | 45EC; 1xSSC, 50% | |
| | | | formamide | |
| D | DNA:RNA | <50 | T_{D}*; 1xSSC | T_{D}*; 1xSSC |
| E | RNA:RNA | >50 | 70EC; 1xSSC -or- | 70EC; 0.3xSSC |
| | | | 50EC; 1xSSC, 50% | |
| | | | formamide | |
| F | RNA:RNA | <50 | T_{F}*; 1xSSC | T_{f}*; 1xSSC |
| G | DNA:DNA | >50 | 65EC; 4xSSC -or- | 65EC; 1xSSC |
| | | | 42EC; 4xSSC, 50% | |
| | | | formamide | |
| H | DNA:DNA | <50 | T_{H}*; 4xSSC | T_{H}*; 4xSSC |
| I | DNA:RNA | >50 | 67EC; 4xSSC -or- | 67EC; 1xSSC |
| | | | 45EC; 4xSSC, 50% | |
| | | | formamide | |
| J | DNA:RNA | <50 | T_{J}*; 4xSSC | T_{J}*; 4xSSC |
| K | RNA:RNA | >50 | 70EC; 4xSSC -or- | 67EC; 1xSSC |
| | | | 50EC; 4xSSC, 50% | |
| | | | formamide | |
| L | RNA:RNA | <50 | T_{L}*; 2xSSC | T_{L}*; 2xSSC |
| M | DNA:DNA | >50 | 50EC; 4xSSC -or- | 50EC; 2xSSC |
| | | | 40EC; 6xSSC, 50% | |
| | | | formamide | |
| N | DNA:DNA | <50 | T_{N}*; 6xSSC | T_{N}*; 6xSSC |
| O | DNA:RNA | >50 | 55EC; 4xSSC -or- | 55EC; 2xSSC |
| | | | 42EC; 6xSSC, 50% | |
| | | | formamide | |
| P | DNA: RNA | <50 | T_{P}*; 6xSSC | T_{P}*; 6xSSC |
| Q | RNA:RNA | >50 | 60EC; 4xSSC -or- | 60EC; 2xSSC |
| | | | 45EC; 6xSSC, 50% | |
| | | | formamide | |
| R | RNA:RNA | <50 | T_{R}*; 4xSSC | T_{R}*; 4xSSC |
| ^{I}: The hybrid length is that anticipated for the hybridized region(s) of the hybridizing polynucleotides. When hybidizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}: SSPE (1xSSPE is 0.15 M NaCl, 10 mM NaH₂PO₄, and 1.25 mM EDTA, pH 7.4) can be substituted for SSC (1xSSC is 0.15 M NaCl and 15 mM sodium citrate) in the hybridization and wash buffers; washes are performed for 15 minutes after hybridization is complete. | | | | |
| *T_{B}-T_{R}: The hybridization temperature for hybrids anticipated to be less than 50 base pairs in length should be 5-10 EC less than the melting temperature (T_{M}) of the hybrid, where T_{M} is determined according to the following equations. For hybrids less than 18 base pairs in length, Tₘ(EC) = 2(# of A + T bases) + 4(# of G + C bases). For hybrids between q8 and 49 base pairs in length, | | | | |
| #Tₘ(EC) = 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1xSSC = 0.165 M). | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Preferably, each such hybridizing polynucleotide has a length that is at least 25% (more preferably at least 50%, and most preferably at least 75%) of the length of the polynucleotide of the present invention to which it hybridizes, and has at least 60% sequence identity (more preferably, at least 75% identity; most preferably at least 90% or 95% identity) with the polynucleotide of the present invention to which it hybridizes, where sequence identity is determined by comparing the sequences of the hybridizing polynucleotides when aligned so as to maximize overlap and identity while minimizing sequence gaps.

Expression of BBPs

The isolated polynucleotide of the invention may be operably linked to an expression control sequence such as the pMT2 or pED expression vectors disclosed in Kaufman et al., Nucleic Acids Res. 19, 4485-4490 (1991), in order to produce the protein recombinantly. Many suitable expression control sequences are known in the art. General methods of expressing recombinant proteins are also known and are exemplified in R. Kaufman, Methods in Enzymology 185, 537-566 (1990). As defined herein “operably linked” means that the isolated polynucleotide of the invention and an expression control sequence are situated within a vector or cell in such a way that the protein is expressed by a host cell which has been transformed (transfected) with the ligated polynucleotide/expression control sequence.

Expression Systems for BBPs

A number of types of cells may act as suitable host cells for expression of the protein. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac7 kit), and such methods are well known in the art, as described in Summers and Smith, Texas Agricultural Experiment Station Bulletin No. 1555 (1987), incorporated herein by reference. As used herein, an insect cell capable of expressing a polynucleotide of the present invention is “transformed.”

The protein of the invention may be prepared by culturing transformed host cells under culture conditions suitable to express the recombinant protein. The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-toyopearl7 or Cibacrom blue 3GA Sepharose7; one or more steps involving hydrophobic interaction chromatography using such resins as phenyl ether, butyl ether, or propyl ether; or immunoaffinity chromatography.

Alternatively, the protein of the invention may also be expressed in a form which will facilitate purification. For example, it may be expressed as a fusion protein, such as those of maltose binding protein (MBP), glutathione-S-transferase (GST) or thioredoxin (TRX). Kits for expression and purification of such fusion proteins are commercially available from New England BioLab (Beverly, Mass.), Pharmacia (Piscataway, N.J.) and InVitrogen, respectively. The protein can also be tagged with an epitope and subsequently purified by using a specific antibody directed to such epitope. One such epitope (“Flag”) is commercially available from Kodak (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The proteins of the invention may also be expressed as a products of transgenic animals, e.g., as a component of the milk of transgenic cows, goats, pigs, or sheep which are characterized by somatic or germ cells containing a nucleotide sequence encoding the protein.

The proteins may also be produced by known conventional chemical synthesis. Methods for constructing the proteins of the present invention by synthetic means are known to those skilled in the art. The synthetically-constructed protein sequences, by virtue of sharing primary, secondary or tertiary structural and/or conformational characteristics with proteins may possess biological properties in common therewith, including protein activity. Thus, they may be employed as biologically active or immunological substitutes for natural, purified proteins in screening of therapeutic compounds and in immunological processes for the development of antibodies.

The proteins provided herein also include proteins characterized by amino acid sequences similar to those of purified proteins but into which modification are naturally provided or deliberately engineered. For example, modifications in the peptide or DNA sequences can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584). Preferably, such alteration, substitution, replacement, insertion or deletion retains the desired activity of the protein.

Other fragments and derivatives of the sequences of proteins which would be expected to retain protein activity in whole or in part and may thus be useful for screening or other immunological methodologies may also be easily made by those skilled in the art given the disclosures herein. Such modifications are believed to be encompassed by the present invention.

Proteins and protein fragments of the present invention include proteins with amino acid sequence lengths that are at least 25% (more preferably at least 50%, and most preferably at least 75%) of the length of a disclosed protein and have at least 60% sequence identity (more preferably, at least 75% identity; most preferably at least 90% or 95% identity) with that disclosed protein, where sequence identity is determined by comparing the amino acid sequences of the proteins when aligned so as to maximize overlap and identity while minimizing sequence gaps. Also included in the present invention are proteins and protein fragments that contain a segment preferably comprising 8 or more (more preferably 20 or more, most preferably 30 or more) contiguous amino acids that shares at least 75% sequence identity (more preferably, at least 85% identity; most preferably at least 95% identity) with any such segment of any of the disclosed proteins.

Species homologues of the disclosed polynucleotides and proteins are also provided by the present invention. As used herein, a species homologue is a protein or polynucleotide with a different species of origin from that of a given protein or polynucleotide, but with significant sequence similarity to the given protein or polynucleotide. Preferably, polynucleotide species homologues have at least 60% sequence identity (more preferably, at least 75% identity; most preferably at least 90% identity) with the given polynucleotide, and protein species homologues have at least 30% sequence identity (more preferably, at least 45% identity; most preferably at least 60% identity) with the given protein, where sequence identity is determined by comparing the nucleotide sequences of the polynucleotides or the amino acid sequences of the proteins when aligned so as to maximize overlap and identity while minimizing sequence gaps. Species homologues may be isolated and identified by making suitable probes or primers from the sequences provided herein and screening a suitable nucleic acid source from the desired species. Preferably, species homologues are those isolated from mammalian species. Most preferably, species homologues are those isolated from certain mammalian species such as, for example, Pan troglodytes, Gorilla gorilla, Pongo pygmaeus, Hylobates concolor, Macaca mulatta, Papio papio, Papio hamadryas, Cercopithecus aethiops, Cebus capucinus, Aotus trivirgatus, Sanguinus oedipus, Microcebus murinus, Mus musculus, Rattus norvegicus, Cricetulus griseus, Felis catus, Mustela vison, Canis familiaris, Oryctolagus cuniculus, Bos taurus, Ovis aries, Sus scrofa, and Equus caballus, for which genetic maps have been created allowing the identification of syntenic relationships between the genomic organization of genes in one species and the genomic organization of the related genes in another species (O'Brien and Seuanez, 1988, Ann. Rev. Genet. 22: 323-351; O'Brien et al., 1993, Nature Genetics 3:103-112; Johansson et al., 1995, Genomics 25: 682-690; Lyons et al., 1997, Nature Genetics 15: 47-56; O'Brien et al., 1997, Trends in Genetics 13(10): 393-399; Carver and Stubbs, 1997, Genome Research 7:1123-1137; all of which are incorporated by reference herein).

The invention also encompasses allelic variants of the disclosed polynucleotides or proteins; that is, naturally-occurring alternative forms of the isolated polynucleotides which also encode proteins which are identical or have significantly similar sequences to those encoded by the disclosed polynucleotides. Preferably, allelic variants have at least 60% sequence identity (more preferably, at least 75% identity; most preferably at least 90% identity) with the given polynucleotide, where sequence identity is determined by comparing the nucleotide sequences of the polynucleotides when aligned so as to maximize overlap and identity while minimizing sequence gaps. Allelic variants may be isolated and identified by making suitable probes or primers from the sequences provided herein and screening a suitable nucleic acid source from individuals of the appropriate species.

The invention also includes polynucleotides with sequences complementary to those of the polynucleotides disclosed herein.

Applications

BBP proteins of the present invention can be used in a variety of applications routine to one of skill in the art based upon this disclosure. Specifically the BBPs can be used as immunogens to raise antibodies which are specific to the cloned polypeptides. Various procedures known in the art may be used for the production of antibodies to BBP proteins. Such antibodies include, but are not limited to, polyclonal, monoclonal, chimeric, single chain, Fab fragments and an Fab expression library. For the production of antibodies, various host animals including, but not limited to rabbits, mice, and rats, are injected with a BBP. In one embodiment, the polypeptide or a fragment of the polypeptide capable of specific immunoactivity is conjugated to an immunogenic carrier. Adjuvants may also be administered in conjunction with the polypeptide to increase the immunologic response of the host animal. Examples of adjuvants which may be used include, but are not limited to, complete and incomplete Freund's, mineral gels such as aluminum hydroxide, surface active substances such as lysolecithin, pluronic polyols, polyanions, peptides, oil emulsions, keyhole limpet hemocyanin, and dinitrophenol.

Monoclonal antibodies to BBP proteins of the present invention can be prepared using any technique which provides for the production of antibodies by continuous cell line in culture. Such techniques are well known to those of skill in the art and include, but are not limited to, the hybridoma technology originally described by Kohler and Milstein (Nature 1975, 256,4202-497), the human B-cell hybridoma technique described by Kosbor et al. (Immunology Today 1983, 4, 72) and the EBV-hybridoma technique described by Cole et al. (Monoclonal Antibodies and Cancer Therapy, Alan R. Liss, Inc., pp 77-96).

Antibodies immunoreactive to the polypeptides of the present invention can then be used to screen for the presence and subcellular distribution of similar polypeptides in biological samples. In addition, monoclonal antibodies specific to the BBP proteins of the present invention can be used as therapeutics.

The BBP proteins can also serve as antigens useful in solid phase assays measuring the presence of antibodies which immunoreact with the claimed peptides. Solid phase competition assays can be used to measure immunological quantities of BBP-related antigen in biological samples. This determination is not only useful in facilitating the complete characterization of the cellular function or functions of the polypeptides of the present inventions, but can also be used to identify patients with abnormal amounts of these proteins.

In addition, these BBPs are useful as reagents in an assay to identify candidate molecules which effect the interaction of BBP and a cloned protein. Compounds that specifically block this association could be useful in the treatment or prevention of various diseases, including but not limited to those involving apoptosis.

These BBPs are also useful in acellular in vitro binding. Acellular assays are extremely useful in screening sizable numbers of compounds since these assays are cost effective and easier to perform than assays employing living cells. Upon disclosure of the polypeptides of the present invention, the development of these assays would be routine to the skilled artisan. In such assays, BBP is labeled. Such labels include, but are not limited to, radiolabels, antibodies, and fluorescent or ultraviolet tags. Binding of a BBP or BBP aggregates is first determined in the absence of any test compound. Compounds to be tested are then added to the assay to determine whether such compounds alter this interaction.

### EXAMPLES

The present invention is further described by the following examples. The examples are provided solely to illustrate the invention by reference to specific embodiments. These exemplifications, while illustrating certain specific aspects of the invention do not portray the limitations or circumscribe the scope of the invention.

Materials and Methods

Molecular cloning. Polymerase chain reactions (PCR) utilized Taq polymerase and reagents supplied by the manufacturer (Perkin Elmer Corp., Norwalk, Conn.). The identification and cloning of the BBP1 cDNA are described elsewhere (see U.S. Ser. No. 09/774,936, filed Jan. 31, 2001). BBP2 and BBP3 cDNA sequences were amplified by the RACE technique using reagents and protocols provided by Clontech Laboratories, Inc. (Palo Alto, Calif.) and gene-specific primers designed from expressed sequence tags assembled from the Genbank database as described in the text. The BBP2 cDNA sequence information from RACE products was utilized to design oligonucleotides to amplify the protein coding region in a single DNA fragment. BBP2 cDNA was amplified from a human brain sample using the PCR primers 5′-TGTGCCCGGG AAGATGGTGC TA [SEQ ID NO:7] (sense) plus 5′-CAGAAAGGAA GACTATGGAA AC [SEQ ID NO:8] (antisense). The PCR conditions were 94° C., 9 min then 32 cycles of 94.5° C., 20 sec; 58° C., 20 sec; 72° C., 60 sec using Clontech's Marathon human brain cDNA. The product was cloned into the pCRII vector (Invitrogen Corp., Carlsbad, Calif.) to generate pOZ359. BBP3 cDNAs were identified during RACE procedures using either Clontech's Marathon placenta or brain cDNA libraries. The sense oligo was Clontech's AP1 primer; the BBP3-specific primer (antisense) had the sequence 5′-CACTCACACC ACATCAACTCTA CG [SEQ ID NO:9]. PCR conditions were as suggested by the library manufacturer (Clontech). The short BBP3 cDNA was cloned into the pCRII vector to generate pOZ350; the longer form was cloned to generate pOZ351.

Northern analyses. Human multiple tissue and cancer cell line mRNA Northern blots and a human mRNA dot blot were obtained from Clontech. Tumor RNA blots were obtained from Invitrogen. The BBP1 probe is described elsewhere (patent application co-owned and co-pending AHP 98126). Briefly, it consisted of sequences beginning at nucleotide 201 and extending through the 3′ untranslated region. BBP2 sequences were isolated from pOZ359 on an EcoRI fragment extending from the vector polylinker to an internal site at position 699. The BBP3 probe consisted of the entire cDNA on an EcoRI fragment from pOZ350. β-actin and ubiquitin DNAs were provided by the blot manufacturers. Radiolabeled probes were produced from these DNAs using a random priming method to incorporate ³²P-dCTP (Pharmacia Biotech, Piscataway, N.J.). Hybridizations were performed per manufacturer's (Clontech) instructions in Express Hyb Solution at 68° C. Blots were washed in 2× SSC (1× SSC is 0.15 M sodium chloride, 0.015 M sodium citrate), 0.05% SDS at room temperature, followed by two washes in 0.1× SSC, 0.1% SDS at 50° C. Dot blots were hybridized at 65° C. overnight, washed five times in 2× SSC, 1% SDS at 65° C., then three times in 0.1× SSC, 0.5% SDS. Hybridization signals were visualized by exposure to Kodak BioMax film.

In situ hybridization. To generate riboprobes for BBP mRNAs, pairs of oligonucleotide primers were designed to amplify a 275 to 300 bp region from the 3′ UTR of each cDNA and, in addition, add the promoter sequences for T7 (sense) and T3 (antisense) polymerase. These primers contained the following sequences: BBP1, 5′-TAATACGACT CACTATAGGG TTAGAAGAAA CAGATTTGAG [SEQ ID NO:10] (forward) and 5′-ATTAACCCTC ACTAAAGGGA CAAGTGGCAA CTTGCCTTTG [SEQ ID NO:11] (reverse); BBP2, 5′-TAATACGACT CACTATAGGG AAGAGCTGCC ATCATGGCCC [SEQ ID NO:12] (forward) and 5′-ATTAACCCTC ACTAAAGGGA AAAGGAAGAC TATGGAAACC [SEQ ID NO:13] (reverse); BBP3, 5′- TAATACGACT CACTATAGGG CCTGGGCCAG TGGCGGGAAG [SEQ ID NO:14] (forward) and 5′-ATTAACCCTC ACTAAAGGGA CACTCACACC ACATCAACTC [SEQ ID NO:15] (reverse). PCR products were gel purified on 1.5% low-melt agarose gels, and bands containing the products were excised, phenol and phenol-chloroform extracted, and ethanol precipitated. Pellets were dried and resuspended in 1× TE buffer (10 mM Tris-HCl, 1 mM EDTA, pH 7.4). Fifty ng of DNA template was used for transcription reactions using (³⁵S)-CTP (New England Nuclear, Boston, Mass.) and the Riboprobe Gemini™ System (Promega, Madison, Wis.).

In situ hybridization histochemistry using sections of cynomolgus monkey (Macaca fascicularis) brain were performed as described previously (Rhodes et al., 1996). Sections were cut at 10 μm on a Hacker-Brights cryostat and thaw-mounted onto chilled (−20° C.) slides coated with Vectabond reagent (Vector Labs, Burlingame, Calif.). All solutions were prepared in dH₂O treated with 0.1% (v/v) diethylpyrocarbonate and autoclaved. Sections were fixed by immersion in 4% paraformaldehyde in PBS (pH 7.4) then immersed sequentially in 2× SSC, dH₂O, and 0.1M triethanolamine, pH 8.0. The sections were then acetylated by immersion in 0.1 M triethanolamine containing 0.25% (v/v) acetic anhydride, washed in 0.2× SSC, dehydrated in 50, 70 and 90% ethanol, and rapidly dried. One ml of prehybridization solution containing 0.9M NaCl, 1 mM EDTA, 5× Denhardt's, 0.25 mg/ml single-stranded herring sperm DNA (GIBCO/BRL, Gaithersburg, Md.), 50% deionized formamide (EM Sciences, Gibbstown, N.J.) in 10 mM Tris, (pH 7.6), was pipetted onto each slide, and the slides incubated for 3 hrs at 50° C. in a humidified box. The sections were then dehydrated by immersion in 50, 70, and 90% ethanol and air dried. Labeled riboprobes were added at a final concentration of 50,000 cpm/μl to hybridization solution containing 0.9M NaCl, 1 mM EDTA, 1× Denhardt's, 0.1 mg/ml yeast tRNA, 0.1 mg/ml single-stranded salmon sperm DNA, dextran sulfate (10%), 0.08% BSA, 10 mM DTT (Boehringer Mannheim, Indianapolis, Ind.), and 50% deionized formamide in 10 mM Tris (pH 7.6). The probes were then denatured at 95° C. (1 min), placed on ice (5 min), and pipetted onto the sections and allowed to hybridize overnight at 55° C. in a humidified chamber. The sections were subsequently washed 1×45 min at 37° C. in 2× SSC containing 10 mM DTT, followed by 1×30 min at 37° C. in 1× SSC containing 50% formamide, and 1×30 min at 37° C. in 2× SSC. Single stranded and non-specifically hybridized riboprobe was digested by immersion in 10 mM Tris pH 8.0 containing bovine pancreas RNAse A (Boehringer Mannheim; 40 mg/ml), 0.5M NaCl, and 1 mM EDTA. The sections were washed in 2× SSC for 1 hr at 60° C., followed by 0.1× SSC containing 0.5% (w/v) sodium thiosulfate for 2 hrs at 60° C. The sections were then dehydrated in 50, 70, 90% ethanol containing 0.3M ammonium acetate, and dried. The slides were loaded into X-ray cassettes and opposed to Hyperfilm b-Max (Amersham) for 14-30 days. Once a satisfactory exposure was obtained, the slides were coated with nuclear-track emulsion (NTB-2; Kodak) and exposed for 7-21 days at 4° C. The emulsion autoradiograms were developed and fixed according to the manufacturer's instructions, and the underlying tissue sections were stained with hematoxylin. To assess nonspecific labeling, a control probe was generated from a template provided in the Riboprobe Gemini™ System kit (Promega). This vector was linearized using Scal and transcribed using T3 polymerase. The resulting transcription reaction generates two products, a 250 base and a 1,525 base riboprobe, containing only vector sequence. This control probe mixture was labeled as described above and added to the hybridization solution at a final concentration of 50,000 cpm/μl. No specific hybridization was observed in control sections, i.e., these sections gave a very weak uniform hybridization signal that did not follow neuroanatomical landmarks (data not shown).

Reverse transcription polymerase chain reaction (RT-PCR). Total RNA was isolated from the cell lines described in the text by the TRIzol method (Life Technologies). 500 ng of each RNA sample was used as template for RT-PCRs using Titan One-Step RT-PCR reagents (Boehringer Mannheim). Primers are listed below.

| | | | |
|---|---|---|---|
| | plus strand | minus strand | Product |
| | primer | primer | length |
| GENE | 5′ to 3′ | 5′ to 3′ | (basepairs) |

| | | | |
|---|---|---|---|
| b-actin | CCCCCATGCCATCC | GACTCGTCATACTC | 581 |
| | TGCGTCTGGA | CTGCTTGCTG | |
| | [SEQ ID NO:16] | [SEQ ID NO:17] | |
| BBP1 | AGATCGATTTTACC | GAGACAGAAGCCCG | 436 |
| | TTGGATACCC | AGAAACACTA | |
| | [SEQ ID NO:18] | [SEQ ID NO:19] | |
| BBP2 | GAATTCATCTCTAC | CACGGCCATTTCTA | 412 |
| | AGGCTCAAAA | TTTCTGCTGA | |
| | [SEQ ID NO:20] | [SEQ ID NO:21] | |
| BBP3 | GCAGCTTCCTGAAA | CACCACATCAACTC | 427 |
| | CAGATTACGA | TACGGACAAA | |
| | [SEQ ID NO:22] | [SEQ ID NO:23] | |

RT-PCRs were performed with the incubations 50-C, 30 min; 94-C, 2 min followed by 32 cycles of 94-C, 25 sec; 52-C (BBP1 and BBP2 reactions) or 58-C (b-actin and BBP3 reactions), 20 sec; 68-C, 40 sec. Eight microliters of each 50 microliter reaction were examined on a 1.8% agarose gel. Each set of reactions included a no template control.

Yeast two-hybrid assays. Y2H expression plasmids were constructed in the vectors pAS2 and pACT2 (Wade Harper et al., 1993). Strain CY770 (Ozenberger and Young, 1995) served as the host for Y2H assays. Sequences encoding the BBP1 intracellular loop were amplified using the oligonucleotides 5′-CCTTCC ATG GAA GTG GCA GTC GCA TTG TCT [SEQ ID NO:24] plus 5′-AACACTCGAG TCA AAA CCC TAC AGT GCA AAA C [SEQ ID NO:25]. This product, containing BBP1 codons 185 to 217, was digested with NcoI+XhoI and cloned into pAS2 cleaved with NcoI+SalI to generate pOZ339. Sequences encoding the BBP2 intracellular loop were amplified using the oligonucleotides 5′-CCATG GCC ACT TTA CTC TAC TCC TTC TT [SEQ ID NO:26] plus 5′-CTCGAG TCA AAT CCC AAG TCC TCC AAG CG [SEQ ID NO:27]. This product, containing BBP2 codons 154 to 188, was cloned into the TA system and then digested with NcoI+XhoI and cloned into pAS2 cleaved with NcoI+SalI to generate pOZ355. Sequences encoding the BBP3 intracellular loop were amplified using the oligonucleotides 5′-CCATG GCT CTG GCT CTA AGC ATC ACC C [SEQ ID NO:28] plus 5′-CTCGAG TCA TAT TCC CAG GCC ACC GAA GC [SEQ ID NO:29]. This product, containing BBP3 codons 163 to 198, was cloned into the TA system and then digested with NcoI+XhoI and cloned into pAS2 cleaved with NcoI+SalI to generate pOZ358. Construction of all Ga protein expression plasmids utilized the BamHI site near the center of each rat cDNA sequence (Kang et al., 1990) as the site of fusion in pACT2. Sense primers annealed to sequences 5′ of the BamHI site; antisense primers annealed to sequences 3′ of the stop codon and included a SalI restriction site. Primers were: Gao, 5′-GTGGATCCAC TGCTTCGAGG AT [SEQ ID NO:30], 5′-GTCGACGGTT GCTATACAGG ACAAGAGG [SEQ ID NO:31]; Gas, 5′-GTGGATCCAG TGCTTCAATG AT [SEQ ID NO:32], 5′-GTCGACTAAA TTTGGGCGTT CCCTTCTT [SEQ ID NO:33]; Gai2, 5′-GTGGATCCAC TGCTTTGAGG GT [SEQ ID NO:34], 5′-GTCGACGGTC TTCTTGCCCC CATCTTCC [SEQ ID NO:35]. PCR products were cloned into the TA vector. G_ sequences were isolated on BamHI-SalI fragments and cloned into pACT2 digested with BamHI+XhoI.

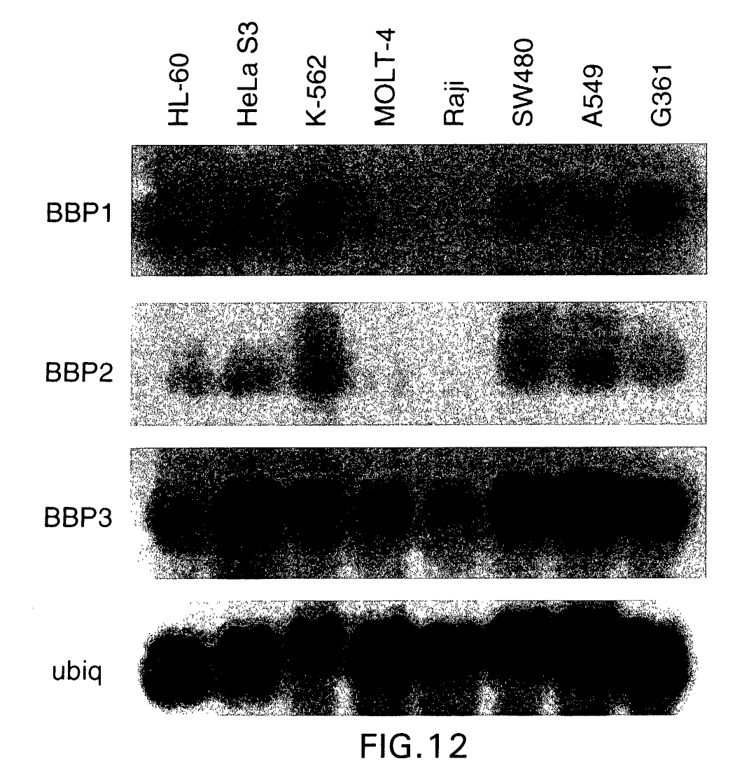

The various combinations of plasmids were transformed into strain CY770 by standard protocols. For bioassays, strains were grown overnight in 2 ml SC medium lacking leucine and tryptophan to a density of approximately 7×10⁷ cells per ml. Cells were concentrated by centrifugation, counted and 10-fold serial dilutions made from 10⁴ to 10⁸ cells per ml in sterile water. These samples were spotted in 5 ml aliquots on SC medium lacking leucine, tryptophan and histidine and containing 25 mM 3-amino-triazole. Plates were incubated at 30° C. for 4 days. Positive protein/protein interactions are identified by increased prototrophic growth compared to control strains expressing the Gal4 DNA-binding domain fusion and containing the pACT vector without inserted sequences. These control strains are indicated in FIGS. 13-15 by the label ‘vector’. This assay method is highly reproducible and provides for the detection of subtle inductions of growth mediated by the specific interaction between target proteins.

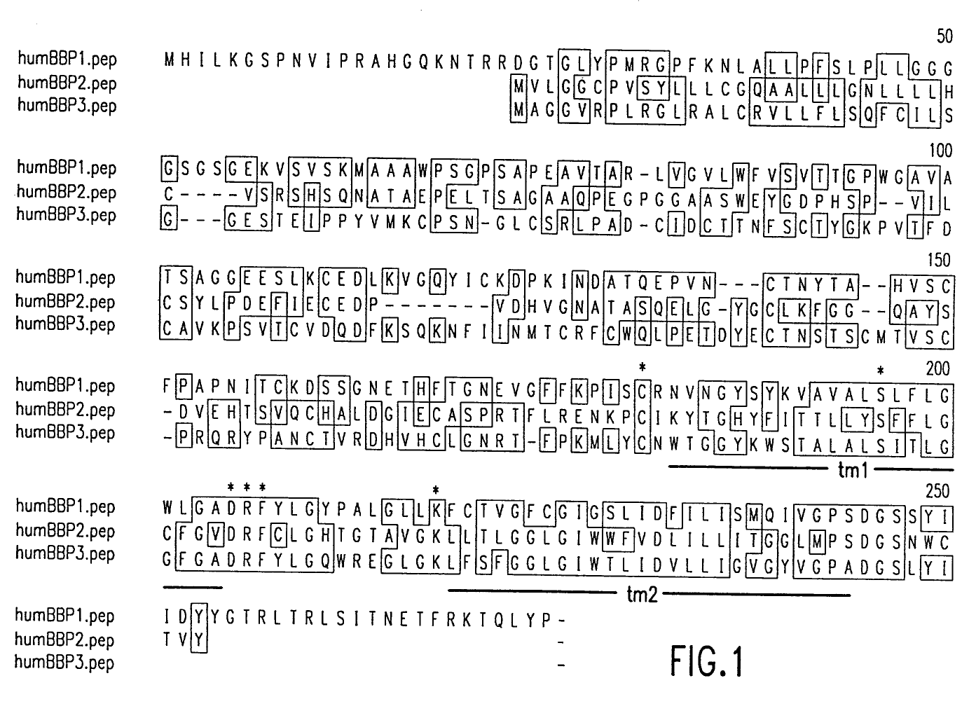

Mammalian expression plasmids. BBP cDNAs were modified by polymerase chain reaction (PCR) for expression from the vector pcDNA3.1 (Invitrogen Corp., Carlsbad, Calif.). BBP1 cDNA was amplified from pBBP1-fl (ATCC #98617); from the third potential translation start site to the translation stop codon, adding a 5′ EcoRI and a 3′ SalI site for cloning. The BBP1 cDNA contains three potential translation starts (codons 1, 30 and 63) The third start site was chosen for the described experiments because the first two potential initiating codons lack appropriate sequence context for efficient translation initiation (see Kozak, 1996), and based on similarities of the protein derived from the third start site with a putative BBP1 orthologue from Drosophila melanogaster (Genbank accession AA941984). FIG. 1 depicts this minimal BBP1 translation product to optimize the alignment with the other BBP subtypes. The PCR primers were 5′- TGGTGAATTC GAAAGTGTCG GTCTCCAAG ATG G [SEQ ID NO:36] (+ strand) and 5′-CTTCGTCGAC TTA TGG ATA TAA TTG CGT TTT TC [SEQ ID NO:37] (− strand). The PCR product was digested with EcoRI +Sall and cloned into pcDNA3.1/EcoRI-XhoI to create pOZ363. BBP2 and BBP3 expression plasmids were similarily engineered. BBP2 was amplified from pOZ359 (ATCC #98851; using primers 5′-TTCCGAATTC AAG ATG GTG CTA GGT GGT TGC CC [SEQ ID NO:38] (+ strand) plus 5′-TTCCCTCGAG TTA GTA AAC AGT GCA CCA GTT GC [SEQ ID NO:39] (− strand). The PCR product was digested with EcoRI+XhoI and cloned into pcDNA3.1/EcoRI-XhoI to create pFL11. BBP3 was amplified from pOZ350 (ATCC #98712 using primers 5′-TTTTGAATTC GCAAG ATG GCG GGA GGG GTG CGC [SEQ ID NO:40] (+ strand) plus 5′-TTGGCTCGAG CTA AAT GTA CAA AGA GCC ATC TG [SEQ ID NO:41] (− strand). The PCR product was digested with EcoRI+XhoI and cloned into pcDNA3.1/EcoRI-XhoI to create pFL12. Mutation of the arginine codon within the ‘DRF’ motif of each BBP cDNA was performed using the QuickChange system (Stratagene Co., La Jolla, Calif.). Oligonucleotides were synthesized and purified by Genosys Biotechnologies, Inc. (The Woodlands, Tex.). The R138 codon of BBP1 in pOZ363 was changed to an alanine codon using the oligonucleotide 5′-GG TTG GGA GCA GAT GCA TTT TAC CTT GGA TAC CC [SEQ ID NO: 42]and its exact reverse complement. The changed nucleotides are underlined. The R138 position of BBP1 in pOZ363 was changed to E using the oligonucleotide 5′-GG TTG GGA GCA GAT GAA TTT TAC CTT GGA TAC CC [SEQ ID NO:43] and its exact reverse complement. The R167 position of BBP2 in pFL11 was changed to E using the oligonucleotide 5′- CTG GGA TGT TTT GGT GTG GAT GA A TTC TGT TTG GGA CAC AC [SEQ ID NO:44] and its exact reverse complement. The R177 position of BBP3 in pFL12 was changed to E using the oligonucleotide 5′- GGT GGG TTT GGA GCA GAC GAA TTC TAC CTG GGC CAG TGG [SEQ ID NO:45] and its exact reverse complement.

Cell culture and transfection. Human Ntera2 (Nt2) stem cells (ATCC #CRL-1973) were maintained in Dulbecco's Modified Eagle's medium (high glucose) supplemented with 10% fetal bovine serum. Expression constructs were introduced into cells by electroporation. The cells were split 1:2 the day before electroporation to ensure exponential growth for maximal survival and efficiency. On the day of electroporation the cells were treated with trypsin and washed two times in phosphate buffered saline (PBS). They were resuspended at 1.3×10⁷ cells per 0.3 ml in RPMI 1640 with 10 mM dextrose and 0.1 mM dithiothriotol. DNA amounts were 7.5 mg subject DNA with 2.5 mg pEGFP-N1 (CLONTECH Laboratories, Palo Alto, Calif.) to monitor transfection. Cells were pre-incubated for 10 mins on ice with DNA, pulsed, and post-incubated for 10 min on ice. A GenePulser instrument (BioRad Corp., Hercules, Calif.) was utilized with a cuvette gap of 0.4 cm, voltage of 0.24 kV, and Capacitance of 960 mF. Cells were plated in standard 6-well plates. Staurosporine was added directly to the cells to a concentration of 100 nM approximately 48 hrs after electroporation. After incubation for 3 hrs, the chromatin-specific dye Hoechst 33342 (Molecular Probes, Inc., Eugene, Oreg.) was added to a concentration of 10 ng/ml. Medium was removed after 10 min and cells were washed with PBS. Cells were then fixed by immersion in PBS containing 4% paraformaldehyde.

Microscopy. Cells were visualized on a Zeiss Axiovert fluorescent microscope fitted with dichroic filters as follows. Hoechst dye visualization utilized excitation at 330 microns, emission at 450; EGFP visualization with excitation at 475, emission at 535. A minimum of 60 transfected (EGFP+) cells were scored per sample. All experiments contained duplicate or triplicate samples.

### Example 1

### Identification of BBPs

The initial human BBP1 clone was obtained by using a yeast 2-hybrid (Y2H) genetic screen developed to identify proteins which interact with human BAP₄₂, a potentially more toxic form of BAP as described in co-owned, co-pending U.S. Ser. No. 09/060,609.

The Genbank database was probed for BBP1-like DNA and protein sequences using the basic local alignment search tool (BLAST; Altschul et al., 1990). All BBP ESTs were extracted from the database and aligned, revealing three distinct sets of DNAs and, therefore, three BBP gene and protein subtypes. All three BBP subtypes are represented in both human and mouse data sets. Exhaustive analysis of the Genbank database failed to identify additional subtypes.

Identification and cloning of the complete protein coding region of the BBP1 gene is described elsewhere in U.S. Ser. No. 09/060,609. All BBP2 and BBP3 ESTs were assembled to form a consensus DNA sequence. In addition, oligonucleotide primers were designed for use in the rapid amplification of cDNA ends (RACE) protocol to identify further 5′ sequences in human brain or placenta samples. Once DNA sequences were fully assembled and confirmed, the longest possible protein coding regions were amplified. The BBP2 cDNA encodes a 214 amino acid protein. There is only one ATG codon near the 5′ end that coincides with the single open reading frame. This ATG is preceded by a stop codon in the same reading frame (data not shown), confirming this ATG as the initiating codon. No stop codon preceded the first ATG in the BBP3 cDNA. The first ATG is shown as the initiating codon but it remains possible that additional 5′ sequences have not been identified. This initiation codon would produce a 221 amino acid protein. An alternatively spliced BBP3 cDNA was identified which would lengthen the protein by 26 residues, adding them between amino acids 30 and 31 of the shorter form. The DNAs depicted in SEQ IDs. 1 through 3 are deposited in the American Type Culture Collection (BBP1, #98617; BBP2, #98851; BBP3-short, #98712 and BBP3-long, #98852).

### Example 2

### Characterization of BBPs to GPCRs

The BBP proteins and translations of available expressed sequence tags were aligned, searched for conserved segments, examined for hydrophobicity indicative of transmembrane segments (Kyte and Doolittle, 1982), and evaluated by the MOST (Tatusov et al., 1994) protein motif search algorithm. These analyses revealed a striking similarity to the G protein-coupled receptor family. Specifically, these analyses indicated that BBPs contain two potential transmembrane (tm) domains near their C-termini (FIG. 1). This segment has primary sequence similarity, and potential structural equivalence to tm domains 3 and 4 of G protein-coupled receptors (GPCRs). Some of the most highly conserved residues in this region of GPCRs were also retained in all three of the BBP proteins (FIG. 1). Based on this conservation, it appears that the BBPs present the short loop between the tm domains to the cytosol, and that both protein termini are located in a lumenal compartment or are extracellular. The predicted cytosolic loop contained the three amino acid motif, aspartate (D) or glutamate followed by arginine (R) and an aromatic residue (Y or F) that is commonly referred to as the DRY sequence. This result suggested that the BBP proteins contained a structural module shared with members of the GPCR superfamily. Specifically, it appears that BBPs retain the critical DRF sequence (FIG. 1), between two predicted tm domains. The N-terminal regions exhibited a much lower degree of similarity (FIG. 1), although common hydrophobic regions near the predicted N-termini score positive in a secretory signal peptide prediction algorithm (Nielsen et al., 1997). This data suggests that BBPs are integral membrane proteins transversing the membrane twice with both termini located extracellularly or within a lumenal compartment.

### Example 3

### Normal Tissue Distribution of BBP mRNA Expression

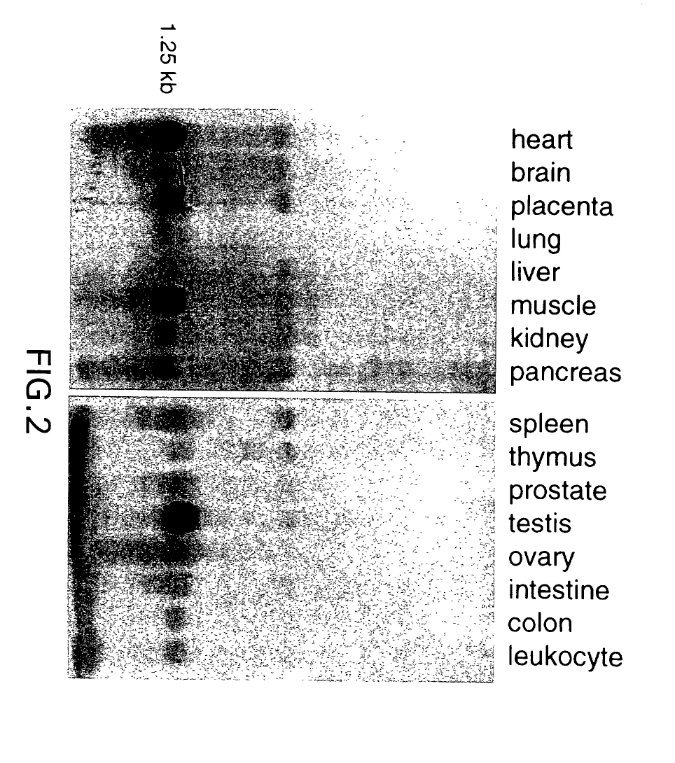

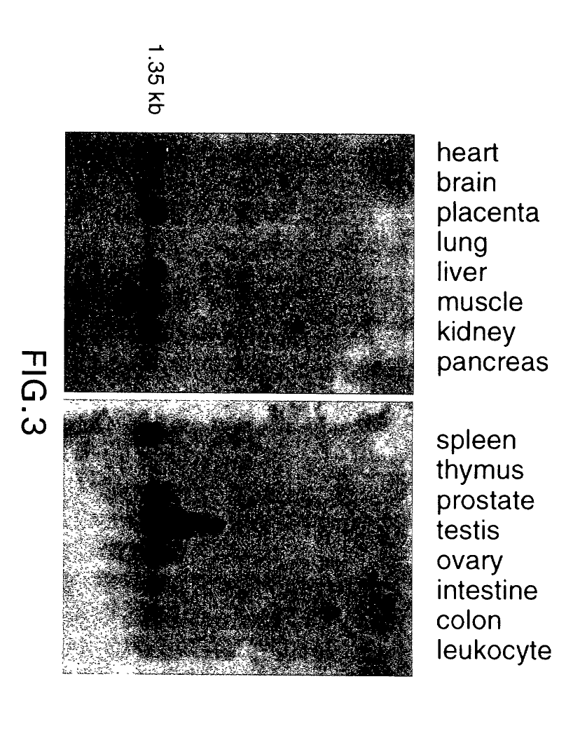

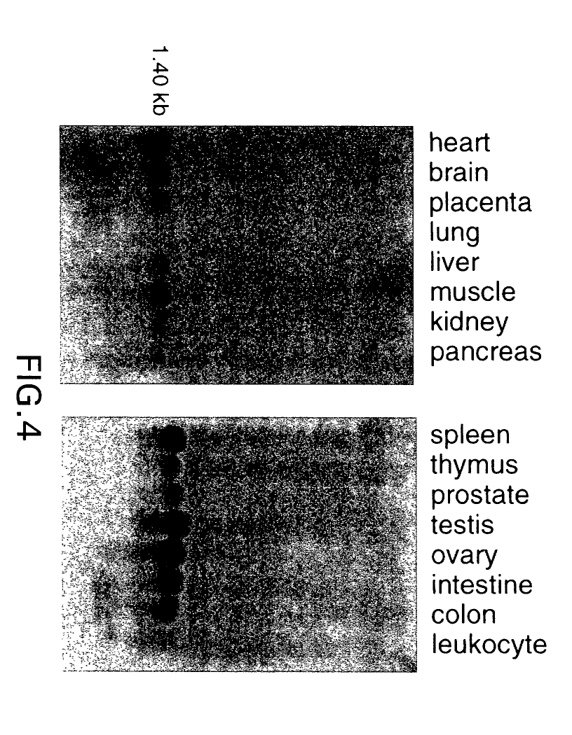

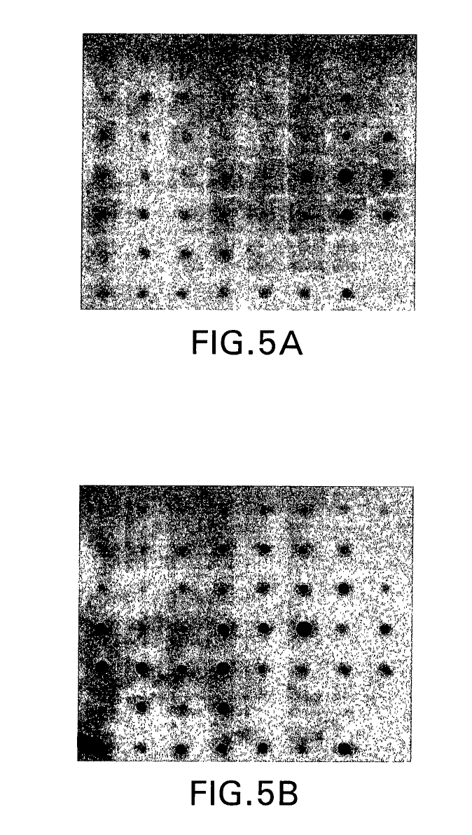

Expression of mRNA in various tissue samples was evaluated as a further step in characterizing the BBP genes. A BBP1 probe revealed a major transcript approximately 1.25 kilobases in length, in all tissues examined (FIG. 2). Higher molecular weight RNAs are likely processing intermediates (i.e., heterogeneous nuclear RNA). BBP2 (FIG. 3) and BBP3 (FIG. 4) probes hybridized to transcripts expressed in all tissues, with sizes of 1.35 and 1.40 kb, respectively. A dot blot of mRNA isolated from 50 different human tissue sources (provided by Clontech Laboratories, Inc., Palo Alto, Calif.) was hybridized with each of the BBP probes to further assess expression patterns. The three BBP genes are expressed in all tissues examined (FIG. 5). There are variations in expression levels (e.g., when comparisons are made between samples and between genes, BBP1 is lower in the cerebellum sample, BBP2 is higher in several glands such as adrenal and thyroid, and BBP3 is more highly expressed in liver), but the conclusion is simply that BBP gene expression is ubiquitous.

### Example 4

### Distribution of BBP mRNA Expression In Brain

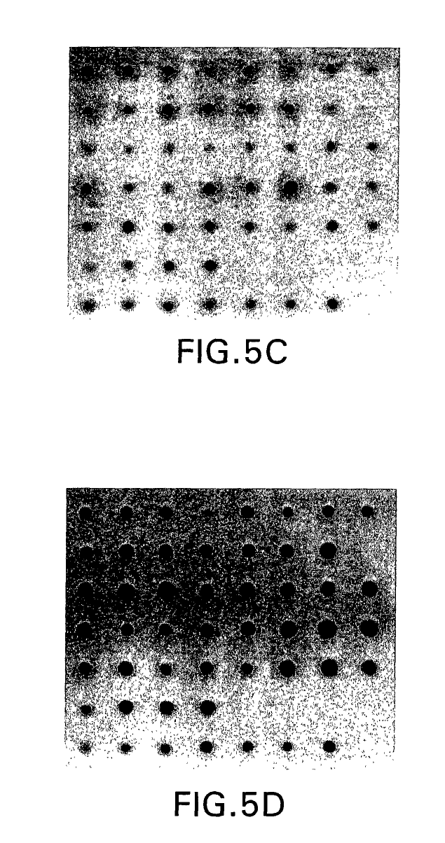

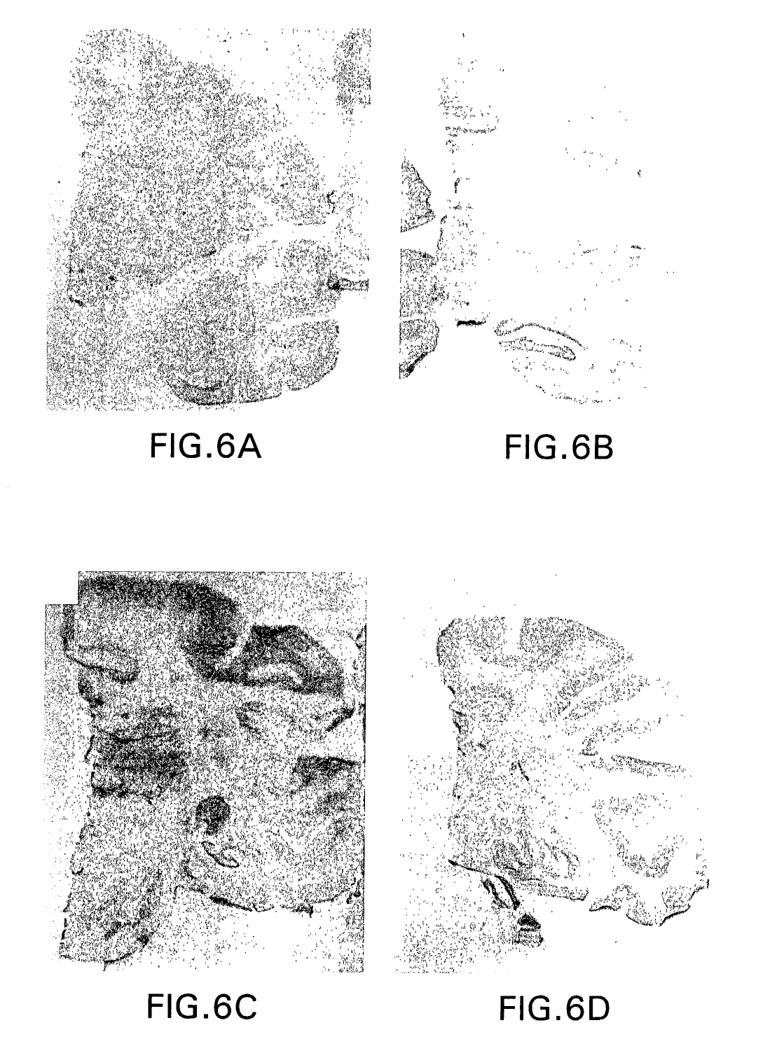

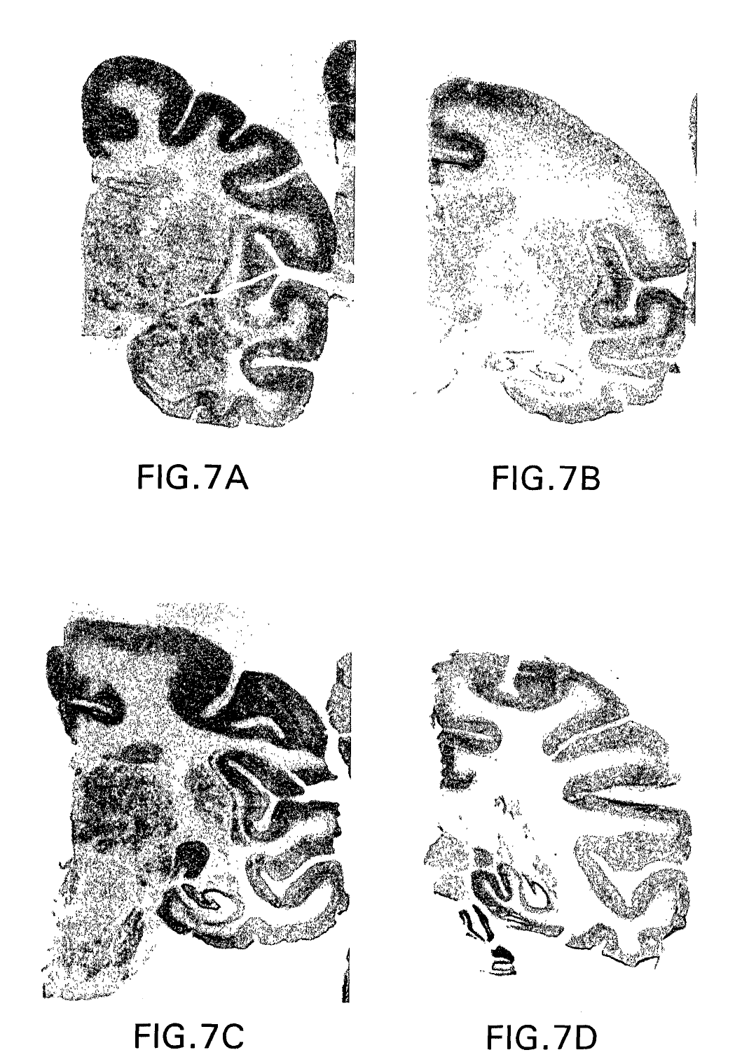

Nonhuman primate (NHP) brain samples were examined by in situ hybridization using BBP subtype-specific riboprobes. BBP1 mRNA was expressed in a pattern consistent with expression in neurons as opposed to glial cells (FIG. 6). There was a greater density of expression in all cortical areas as compared to subcortical structures. The rank order of expression was hippocampus=neocortex=lateral geniculate nucleus>amygdala>>>striatum>thalamus, midbrain and brainstem. BBP2 mRNA was also widely expressed in NHP brain in a pattern consistent with expression in neurons as opposed to glial cells (FIG. 7). The rank order of expression was hippocampus=neocortex=lateral geniculate nucleus=amygdala>striatum=thalamus, midbrain and brainstem. BBP3 mRNA was also widely expressed in NHP brain in a pattern consistent with expression in neurons as opposed to glial cells (FIG. 8). The rank order of expression was hippocampus>neocortex=lateral geniculate nucleus=amygdala>striatum>thalamus, midbrain and brainstem. The pattern and relative density of expression in cortex of all three BBP genes showed considerable overlap. In neocortical areas, there was laminar differentiation that is most striking in limbic and multimodal sensory association cortices. In summary, the BBP genes were widely expressed in NHP brain, with greatest expression in neuronal cells, suggesting activity in a variety of brain processes.

### Example 5

### Distribution of BBP mRNA Expression In Tumors

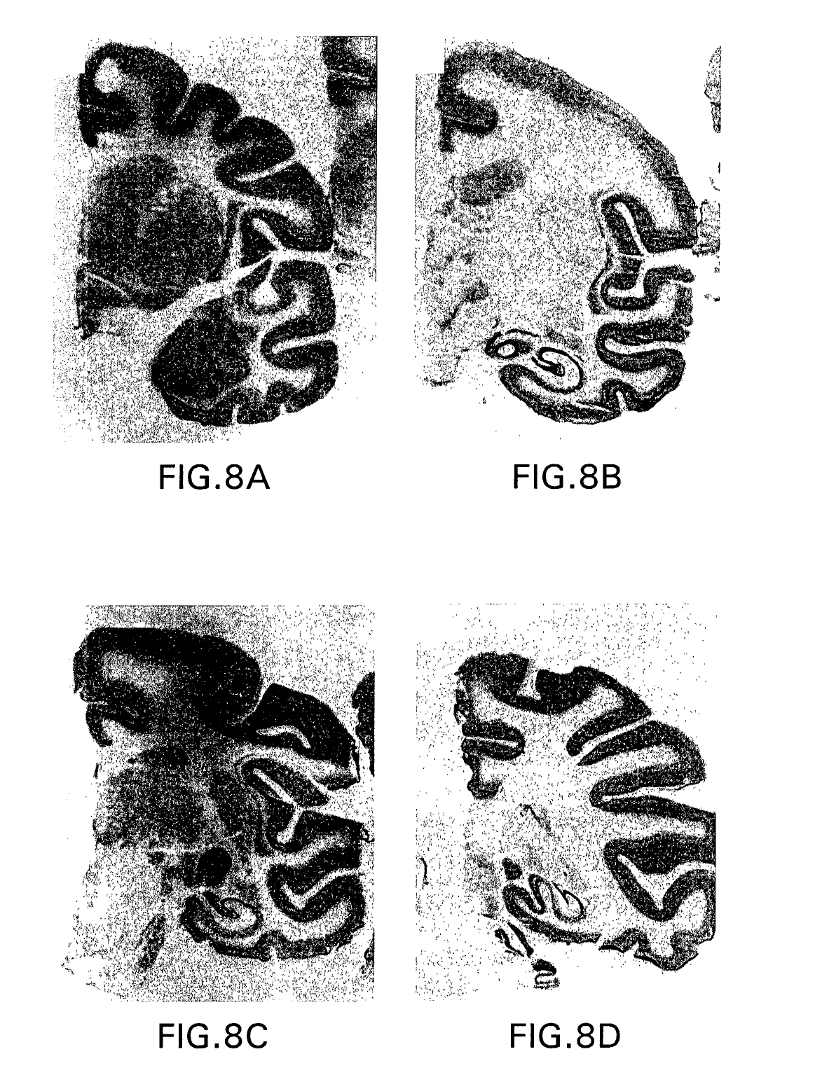

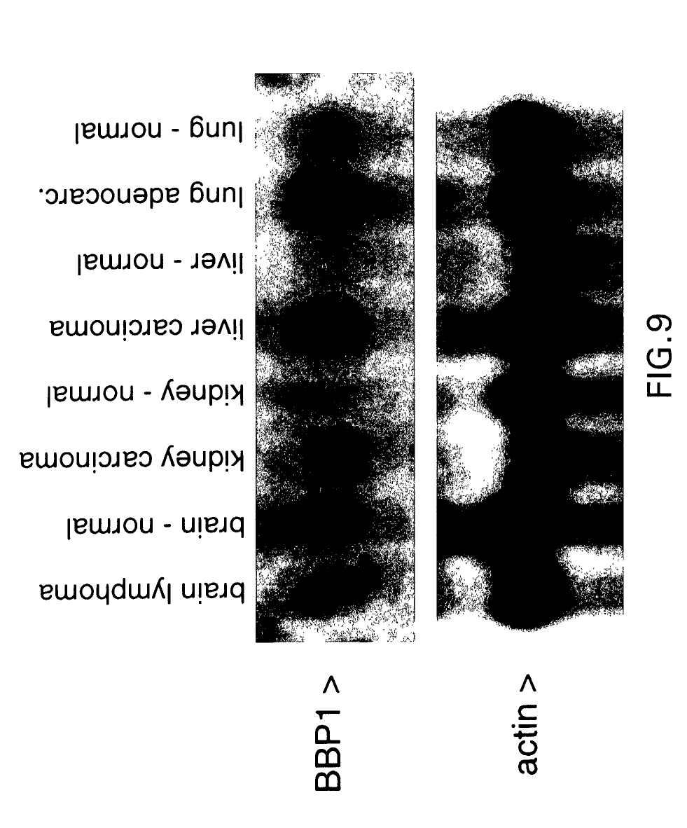

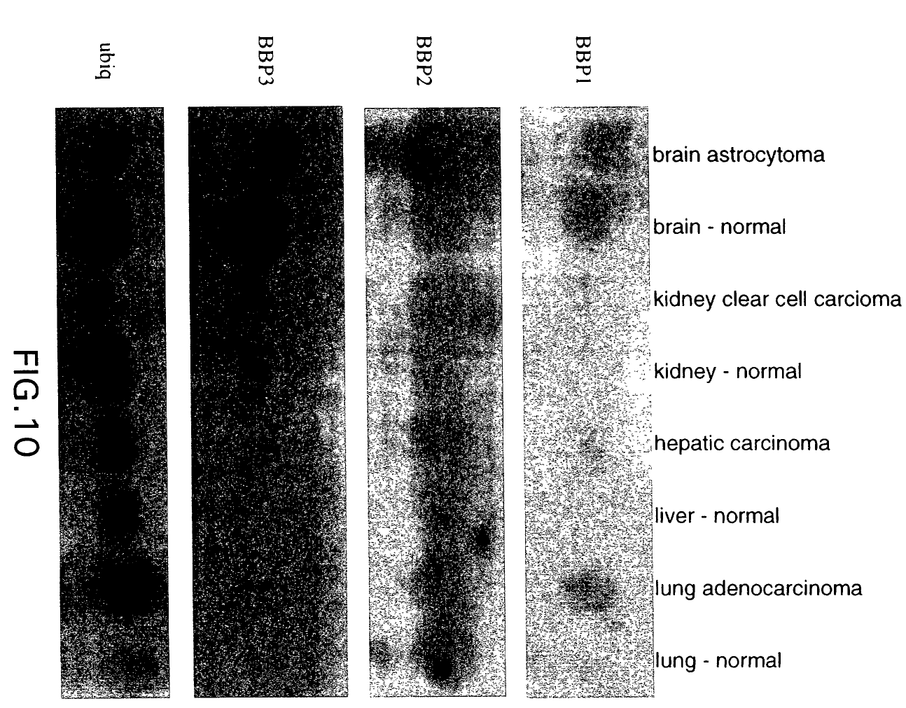

A Northern blot of mRNA isolated from normal and tumor tissue samples was probed with BBP1. This experiment demonstrated that BBP1 was expressed at higher levels in three (kidney, liver, lung) of four tumors examined (FIG. 9). These experiments were extended to include additional tumors and the BBP2 and BBP3 subtypes. Brain astrocytoma, kidney carcinoma, hepatic carcinoma, lung adenocarcinoma, breast carcinoma, uterine leiomyoma, fallopian tube carcinoma, and ovarian thecoma samples were compared to normal tissue samples. BBP1 was overexpressed in the kidney, liver, lung and uterine tumors; BBP2 in brain, breast and uterine tumors; BBP3 in liver, breast and uterine tumors (FIG. 10 and FIG. 11). BBP1 appeared to be underrepresented in the ovarian tumor, and BBP3 in the fallopian tube and ovarian tumors (FIG. 11). These data suggest that all three BBP genes are overexpressed in some tumors, and may therefore, have a function in cellular signaling pathways gating proliferation or death decision points.

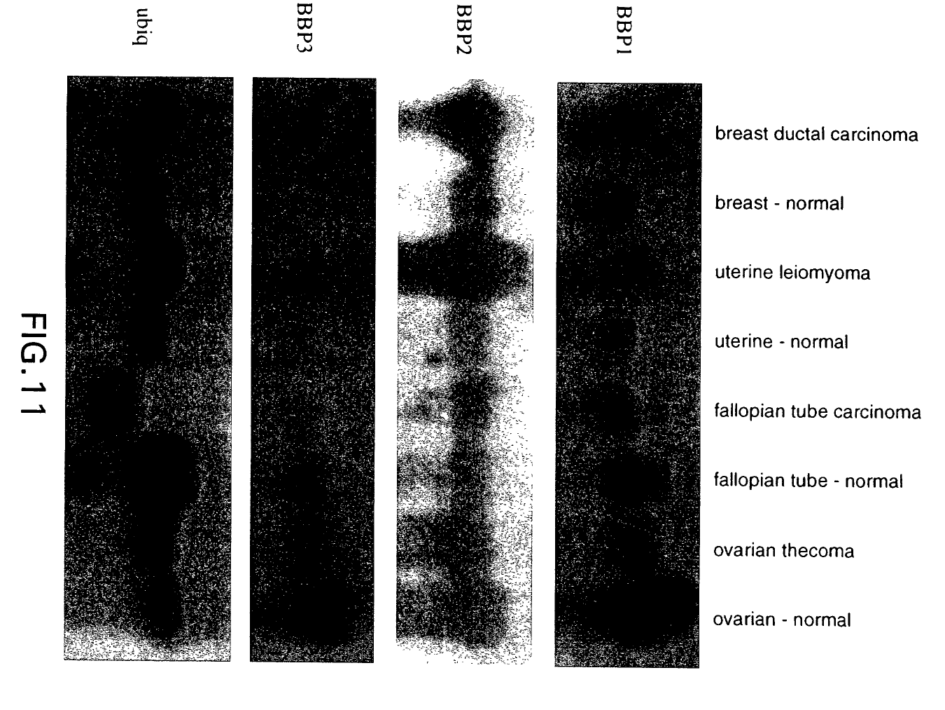

BBP gene expression was also investigated in numerous cancer cell lines and data were extracted from the National Cancer Institute's evaluation of gene expression patterns in the Cancer Genome Anatomy Project. The latter data are available in the National Center for Biotechnology Information's Genbank database (dbEST) of expressed sequence tags (ESTs). Each BBP sequence was used to probe dbEST by BLAST. Those ESTs derived from tumor samples are listed in Table 1. In summary, all three BBP subtypes were present in the Cancer Genome Anatomy Project. Reverse-transcription polymerase chain reaction (RT-PCR) methods were utilized to qualitatively assess BBP mRNA expression in a variety of cancer cell lines. The quantity of RT-PCR product was presented as 0 or 1, 2 or 3 plusses (Table 2). Although these experiments were designed to normalize PCR conditions for each probe, no rigorous quantitative comparisons are implied. BBP mRNAs were observed in all samples in which the positive control b-actin could also be detected, and even in some samples where the control was not detected (Table 2). A Northern blot of eight different cancer cell line samples was probed with BBP subtype-selective probes and ubiquitin as a positive control. Again, all three BBP genes were expressed in all cell lines, although BBP1 and BBP2 were expressed at very low levels in the lymphoblastic leukemia MOLT-4 and Burkitt's lymphoma Raji lines (FIG. 12). The expression of BBP genes in cancer cell lines and the finding that their expression is induced in some tumors suggest that BBP proteins may have activities modulating cell survival and proliferation.

TABLE 1
BBP expressed sequence tags (ESTs) identified in the
National Cancer Institute's Cancer Genome Anatomy Project. The
Genbank dbEST database was probed with each BBP cDNA sequence
by BLAST and those ESTs annotated as originating from tumors were
extracted. This list was last updated on Sep. 23, 1998.

| BBP subtype | tumor type | Accession number |
|---|---|---|
| BBP1 | colon | AA306979 |
| | colon | AA639448 |
| | uterus | AA302858 |
| | prostate | AA613897 |
| | Ewing's sarcoma | AA648700 |
| | parathyroid adenoma | AA772225 |
| | lung | AA975953 |
| | germ cell tumor | AI014369 |
| BBP2 | pancreatic | AA312966 |
| | sarcoma | AA527643 |
| | colon | AA613058 |
| | kidney (clear cell) | AA873687 |
| | lung | AA953791 |
| | breast | AA989378 |
| BBP3 | testis | AA301260 |
| | adrenal | AA319561 |

TABLE 2
BBP mRNA expression in cancer cell lines. Total RNA
from the indicated cancer cell lines was used as template for Rt-PCR
reactions using BBP subtype-selective primers or control -actin primers.
All primers had similar annealing properties and all products were
approximately the same length.

| | -actin | BBP1 | BBP2 | BBP3 |
|---|---|---|---|---|
| Colon | | | | |
| Cx-1 | 0 | + | ++ | + |
| Colo2O5 | + | ++ | ++ | ++ |
| MIP 101 | ++ | ++ | ++ | ++ |
| SW 948 | + | ++ | ++ | ++ |
| CaCo | ++ | + | ++ | + |
| HCT-15 | + | 0 | + | + |
| SW 620 | ++ | ++ | ++ | + |
| LS174T | 0 | + | + | + |
| Ovarian | | | | |
| HTB161 | 0 | 0 | + | 0 |
| A2780 S | ++ | +++ | ++ | ++ |
| A2780 DDp | ++ | +++ | ++ | ++ |
| Breast | | | | |
| MCF-7 | ++ | + | + | + |
| SKBr-3 | ++ | +++ | ++ | ++ |
| T47-D | ++ | +++ | +++ | ++ |
| B7474 | ++ | +++ | +++ | ++ |
| Lung | | | | |
| Lx-1 | + | ++ | ++ | ++ |
| A5439 | + | + | ++ | + |
| Melanoma | | | | |
| Lox | 0 | + | ++ | + |
| SKmeI30 | ++ | ++ | ++ | + |
| Leukemia | | | | |
| HL60 | ++ | ++ | ++ | + |
| CEM | ++ | ++ | ++ | ++ |
| Prostate | | | | |
| LNCAP | + | + | ++ | + |
| Du145 | ++ | ++ | ++ | + |
| PC-3 | + | + | ++ | + |

Key: 0, no RT-PCR product detected; +, any detectable product; ++, large relative amount of product; +++, exceptionally large amount of product.

### Example 6

### BBP Interactions with G Proteins

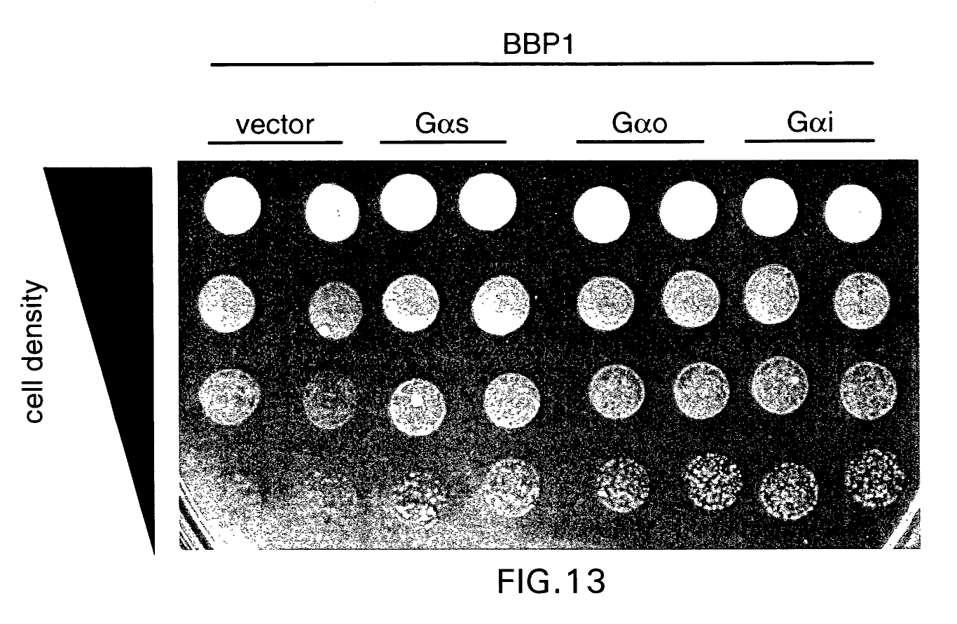

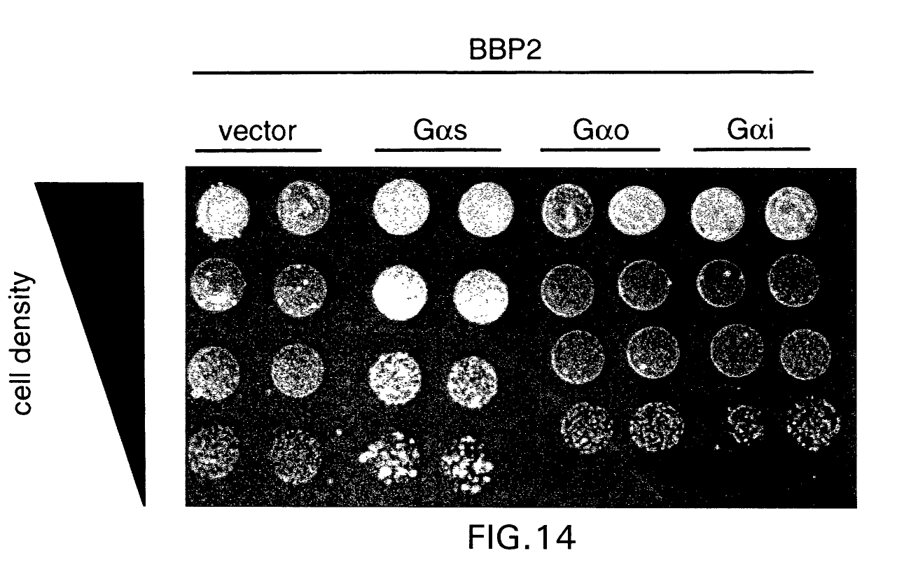

Amyloid precursor protein APP has been shown to functionally associate with the Gαo protein (Nishimoto et al., 1993; Yamatsuji et al., 1996). BBP1 contains a structural motif known to be a Gα protein activating sequence in the related G protein-coupled receptors. The intracellular sequences of each BBP were expressed as fusion proteins and assayed for physical interactions with fusion proteins containing C-terminal regions of Gα proteins in Y2H assays. The BBP1 intracellular loop interacted with all three Gα proteins (FIG. 13). The BBP2 intracellular loop demonstrated preferential interactions with Gαs, exhibiting no apparent association with Gαo or Gαi (FIG. 14). BBP3 also showed a strong response with Gαs (FIG. 15). Additionally, BBP3 exhibited interaction with Gαi, but none with Gαo (FIG. 15). These results demonstrate that the BBP proteins can physically interact with Gα proteins suggesting a possible model of a multiple protein complex potentially composed of integral membrane BBP and APP proteins coupled to heterotrimeric G proteins.

### Example 7

### Suggestive Apoptotic Activity of BBPs

The BBP proteins were examined for effects on cell viability in a robust assay in which the compound staurosporine was used to induce cell death. At the concentration used, staurosporine treatment generally results in rapid biochemical and morphological changes suggestive of apoptosis (Boix et al., 1997; Prehn et al., 1997). The term “apoptosis” is used herein to indicate the appearance of condensed nuclei, a commonly utilized early indicator of apoptosis induction.

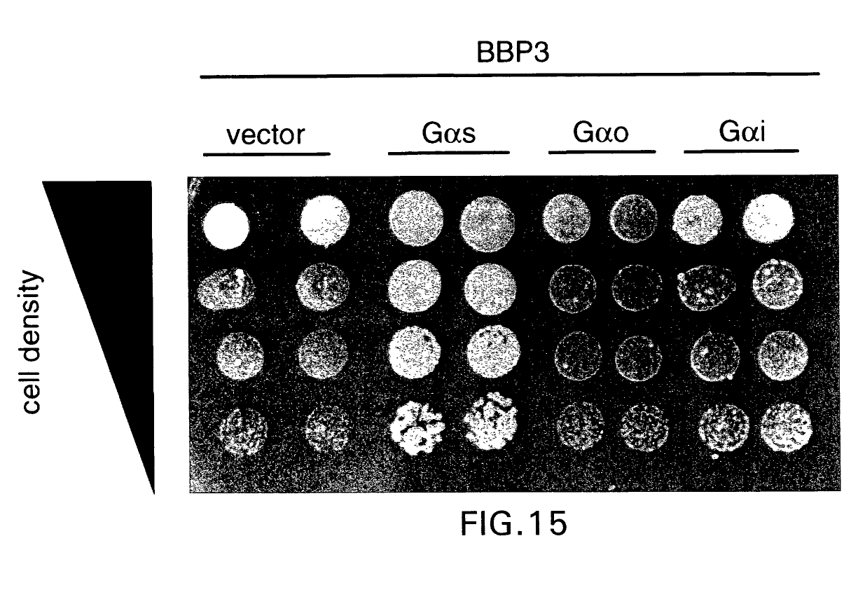

BBP1 effects on cell sensitivity to staurosporine challenge were investigated by cotransfecting the BBP1 expression plasmid pOZ363 plus pEGFP-N1 in human Ntera-2 (Nt2) stem cells at a 3:1 ratio. Expression of green fluorescent protein from pEGFP served as an indicator of cell transfection. Cells were subsequently treated with staurosporine, a potent inducer of apoptosis. Nuclei were revealed by staining with Hoechst 33342, and the frequency of apoptotic transfectants was determined visually by fluorescent microscopy (transfectants are GFP+, apoptotic cells have condensed nuclei). In these assays, cells expressing recombinant BBP1 were protected from apoptosis, exhibiting only 13.5% apoptosis versus 45% for controls (FIG. 16). Expression of a 7-tm domain G protein-coupled serotonin receptor had no effect in the assay (5HT-R, FIG. 16). Throughout these studies, the frequency of condensed nuclei in the absence of inducer (e.g., columns 1-3; FIG. 16) remained fairly constant regardless of experiment, suggesting that the basal level is unrelated to the specific biochemical mechanisms of apoptosis, or that any potential effects on baseline are beyond the sensitivity of the assay system. Expression of recombinant BBP1 not only suppressed nuclear condensation, but also blocked cell death induced by staurosporine, as transfectants with normal nuclear morphology and overall appearance were still observed after a 24 hr treatment with staurosporine, at which point the majority of untransfected or control cells had perished (data not shown).

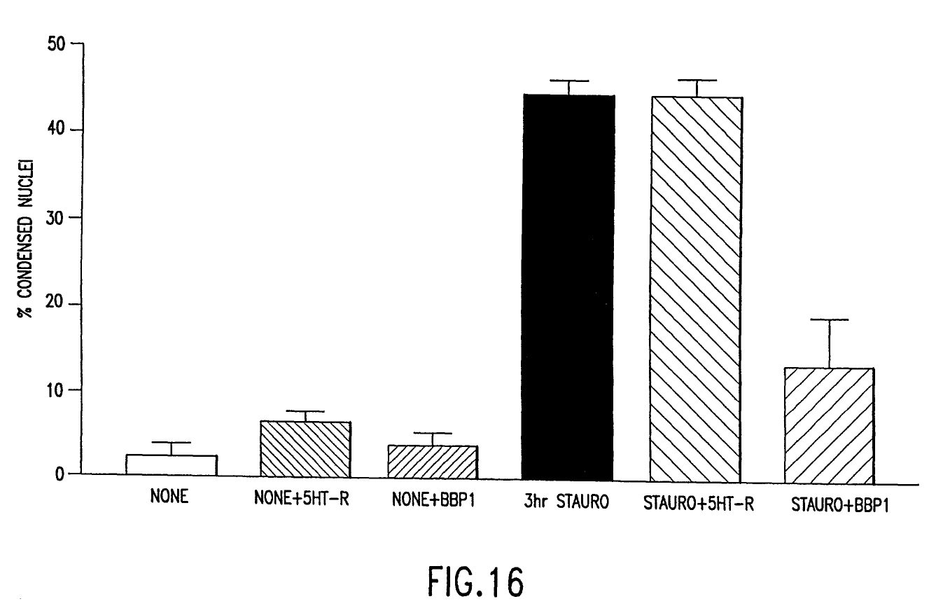

To investigate the potential involvement of G proteins in these events, the arginine in the BBP1 ‘DRF’ motif was replaced by either alanine or glutamate by oligonucleotide-directed mutation of the arginine-138 codon. It is known from studies on members of the 7-tm domain G protein-coupled receptor superfamily that the R to A substitution results in a substantial loss in potential G protein activation, and the R to E substitution generally results in a completely inactive receptor as measured by agonist-induced activation of G protein (Jones et al., 1995; van Rhee and Jacobsen, 1996). The BBP1 mutants failed to suppress apoptosis to the levels of wild-type protein (FIG. 17). The degree of loss of antiapoptotic activity was stepwise and consistent with the known effects on GPCRs (R-A, partial loss; R-E, almost complete loss), suggesting that the results are due to changes in activity rather than protein stability. Substitutions at the same positions in GPCRs has no effect on protein stability or localization (Jones et al., 1995; Rosenthal et al., 1993). The data suggest that BBP1 may integrate with apoptotic signaling pathways via heterotrimeric G protein signal transducers.

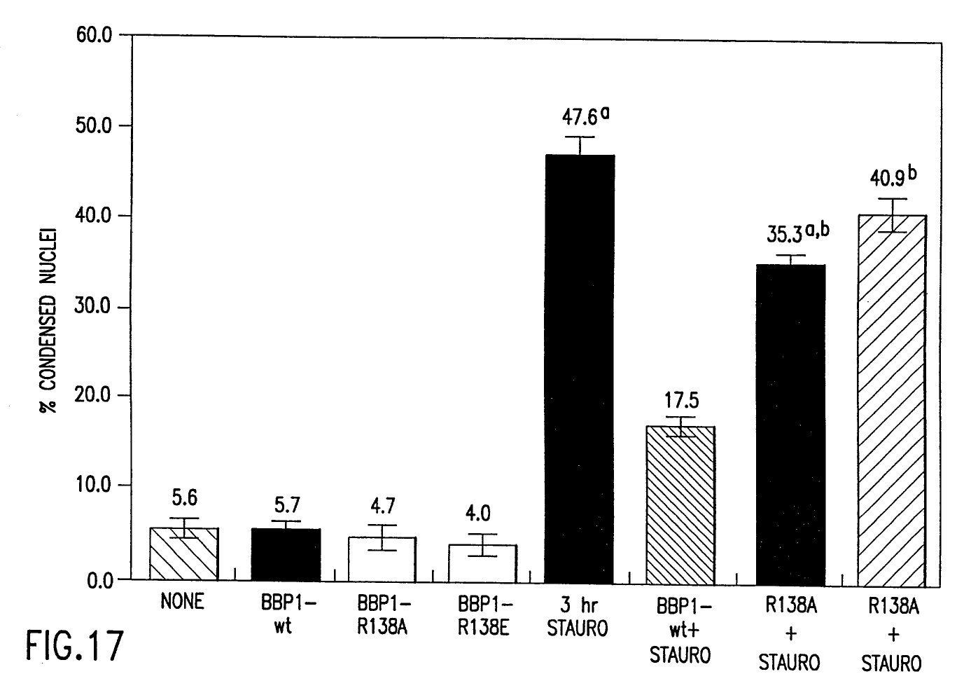

Plasmids (pFL11 and pFL12, respectively) were constructed to express BBP2 or BBP3 in the apoptosis assay system. Expression of these proteins in Nt2 stem cells suppressed the induction of nuclear condensation to the same levels as BBP1 (FIG. 18), demonstrating that each of these structurally related proteins can suppress staurosporine-induced apoptosis. The R to E substitution in the ‘DRF’ motif was engineered in BBP2 and BBP3. This amino acid substitution substantially reduced the antiapoptotic activity of both proteins (FIGS. 22 and 23), again suggesting involvement of heterotrimeric G proteins, which previously were shown to physically associate with the BBP proteins (FIGS. 16-18).

It is clear that the invention may be practiced otherwise than as particularly described in the foregoing description and examples. Numerous modifications and variations of the present invention are possible in light of the above teachings and therefore are within the scope of the appended claims.

### REFERENCES

Acharya, S., and Karnik, S. (1996). Modulation of GDP release from transducin by the conserved Glu134-Arg135 sequence in rhodopsin. J Biol Chem 271, 25406-25411.

Altschul, S., Gish, W., Miller, W., Myers, E., and Lipman, D. (1990). Basic local alignment search tool. J Mol Biol 215, 403-410.

Boix, J., Llecha, N., Yuste, V. -J., and Comella, J. X. (1997). Characterization of the cell death process induced by staurosporine in human neuroblastoma cell lines. Neuropharmacol 36, 811-821.

Jones, P. G., Curtis, C. A. M., and Hulme, E. C. (1995). The function of a highly-conserved arginine residue in activation of the muscarinic M1 receptor. Eur J Pharmacol 288, 251-257.

Kang, Y. -S., Kane, J., Kurjan, J., Stadel, J., and Tipper, D. (1990). Effects of expression of mammalian Ga and hybrid mammalian-yeast Ga proteins on the yeast pheromone response signal transduction pathway. Mol Cell Biol 10, 2582-2590.

Kozak, M. (1996). Interpreting cDNA sequences: some insights from studies on translation. Mammal Genome 7, 563-574.

Kyte, J., and Doolittle, R. (1982). A simple method for displaying the hydropathic character of a protein. J Mol Biol 157, 105-132.

Manning, F., and Patierno, S. (1996). Apoptosis: inhibitor or instigator of carcinogenesis? Cancer Investig 14, 455-465.

Nielsen, H., Engelbrecht, J., Brunak, S., and von Heijne, G. (1997). Identification of prokaryotic and eukaryotic signal peptides and prediction of their cleavage sites. Prot Engineering 10, 1-6.

Nishimoto, I., Okamoto, T., Matsuura, Y., Takahashi, S., Okamoto, T., Murayama, Y., and Ogata, E. (1993). Alzheimer amyloid protein precursor complexes with brain GTP-binding protein Go. Nature 362, 75-79.

Ozenberger, B., and Young, K. (1995). Functional interaction of ligands and receptors of the hematopoietic superfamily in yeast. Mol Endocrinol 9,1321-1329.

Prehn, J. H. M., Jordan, J., Ghadge, G. D., Preis, E., Galindo, M. F., Roos, R. P., Kriegistein, J., and Miller, R. J. (1997). Ca2+ and reactive oxygen species in staurosporine-induced neuronal apoptosis. J Neurochem 68, 1679-1685.

Rhodes, K., Monaghan, M., Barrezueta, N., Nawoschik, S., Bekele-Arcuri, Z., Matos, M., Nakahira, K., Schechter, L., and Trimmer, J. (1996). Voltage-gated K+channel beta subunits: expression and distribution of Kv beta 1 and Kv beta 2 in adult rat brain. J Neurosci 16, 4846-4860.

Rosenthal, W., Antaramian, A., Gilbert, S., Birnbaumer, M. (1993). Nephrogenic diabetes insipidus. A V2 vasopressin receptor unable to stimulate adenylyl cyclase. J Biol Chem 268, 13030-13033.

Selkoe, D. (1997). Alzheimer's Disease: Genotypes, phenotype, and treatments. Science 275, 630-631.

Tatusov, R., Altschul, S., and Koonin, E. (1994). Detection of conserved segments in proteins: Iterative scanning of sequence databases with alignment blocks. Proc Natl Acad Sci U.S. Pat. No. 91, 12091-12095.

Thompson, J., Higgins, D., and Gibson, T. (1994). CLUSTAL W: improving the sensitivity of progressive multiple sequence alignment through sequence weighting, position-specific gap penalties and weight matrix choice. Nucleic Acids Res 22, 4673-4680.

van Rhee, A. M. and Jacobsen, K. A. (1996). Molecular architecture of G protein-coupled receptors. Drug Develop Res 37, 1-38.

Wade Harper, J., Adami, G. R., Wei, N., Keyomarsi, K., and Elledge, S. J. (1993). The p21 Cdk-interacting protein Cip1 is a potent inhibitor of G1 cyclin-dependent kinases. Cell 75, 805-816.

Yamatsuji, T., Matsui, T., Okamoto, T., Komatsuzaki, K., Takeda, S., Fukumoto, H., lwatsubo, T., Suzuki, N., Asami-Odaka, A., Ireland, S., Kinane, T., Giambarella, U., and Nishimoto, I. (1996). G protein-mediated neuronal DNA fragmentation induced by familial Alzheimer's Disease-associated mutants of APP. Science 272,1349-1352.

What is claimed is:

1. A human G protein-coupled receptor like (GPCR-like) protein comprising:
a) an integral membrane protein consisting of two transmembrane domains wherein said transmembrane domains have greater than 95% sequence similarity to transmembrane domains 3 and 4 of a G protein-coupled (GPCR-like); and
b) a short loop between the two transmembrane domains said loop comprising a three amino acid sequence having greater than 95% similarity to a short loop of a GPCR.

2. The GPCR-like protein of claim 1, wherein the short loop between the two transmembrane domains comprises a three amino acid fragment wherein the first amino acid is aspartate or glutamate; the second amino acid is arginine; and the third amino acid is tyrosine or phenylalanine.

3. An isolated nucleic acid comprising the nucleotide sequence of SEQ ID NO: 1 or a degenerate variant of SEQ ID NO: 1.

4. An isolated nucleic acid comprising a sequence that encodes a polypeptide with the amino acid sequence of SEQ ID NO: 2.

5. An isolated nucleic acid comprising the nucleotide sequence of SEQ ID NO: 3 or a degenerate variant of SEQ ID NO: 3.

6. An isolated nucleic acid comprising a sequence that encodes a polypeptide with the amino acid sequence of SEQ ID NO: 4.

7. An isolated nucleic acid comprising the nucleotide sequence of SEQ ID NO: 5 or a degenerate variant of SEQ ID NO: 5.

8. An isolated nucleic acid comprising a sequence that encodes a polypeptide with the amino acid sequence of SEQ ID NO: 6.

9. An isolated nucleic acid comprising a sequence that encodes a polypeptide with the amino acid sequence of GPCR-like protein of clone pOZ359 deposited under accession number ATCC 98851.

10. An isolated nucleic acid comprising a sequence that encodes a polypeptide with the amino acid sequence of GPCR-like protein of clone pOZ350 deposited under accession number ATCC 98712.

11. An isolated nucleic acid comprising a sequence that encodes a polypeptide with the amino acid sequence of GPCR-like protein of clone pOZ351 deposited under accession number ATCC 98852.

12. A polynucleotide comprising at least one expression control sequence operably linked to a polynucleotide selected from the group consisting of the nucleic acids of claims 3,4, 5, 6, 7, 8, 9, 10, 11 or 12.

13. A host cell transformed with the polynucleotide of claim 12.

14. The host cell of claim 13 wherein said cell is a prokaryotic or eukaryotic cell.

15. A method for determining a polynucleotide encoding the GPCR-like protein of claim 1 in a sample comprising the steps of:
(a) hybridizing to a sample a probe specific for said polynucleotide under conditions effective for said probe to hybridize specifically to said polynucleotide; and
(b) determining the hybridization of said probe to polynucleotides in the sample, wherein said probe comprises a nucleic acid sequence having a region of 20 or more base pairs at least 90% identical to the sequences of claims 3, 4, 5, 6, 7, 8, 9, 10, 11 or 12.

16. A method for determining a polynucleotide encoding a GPCR-like protein of claim 1 in a sample comprising the steps of:
(a) hybridizing to a sample a probe specific for said polynucleotide under conditions effective for said probe to hybridize specifically to said polynucleotide; and
(b) determining the hybridization of said probe to polynucleotides in the sample, wherein said probe comprises a nucleic acid sequence having a region of 20 or more base pairs at least 90% identical to the polynucleotide sequence of the cDNA insert of ATCC 98851 or ATCC 98712 or ATCC 98852.

17. A method for detecting in a sample a polypeptide comprising a region at least 90% identical to the amino acid sequence of SEQ ID NOs: 2, 4 or 6 said method comprising:
(a) incubating with a sample a reagent that bind specifically to said polypeptide under conditions effective for specific binding; and
(b) determining the binding of said reagent to said polypeptide the sample.

18. A method for detecting in a sample a polypeptide comprising a region at least 90% identical in sequence to the amino acid sequence of the GPCR-like protein encoded by the cDNA insert of the deposit comprising ATCC 98851, ATCC 98712, or ATCC 98852, said method comprising:
(a) incubating with a sample a reagent that bind specifically to said polypeptide under conditions effective for specific binding; and
(b) determining the binding of said reagent to said polypeptide the sample.

19. A method for demonstrating suppression of nuclear condensation as a measure of staurosporine induced apoptosis in cell culture comprising:
(a) incubating a cell sample undergoing nuclear condensation with a reagent comprising a polypeptide comprising a region at least 90% identical to the amino acid sequence of SEQ ID NO: 2, 4 or 6; and
(b) determining the suppression of induction of nuclear condensation in the sample compared to control containing staurosporine only.

20. A method for demonstrating suppression of nuclear condensation as a measure of staurosporine induced apoptosis in cell culture comprising:
(a) incubating a cell sample undergoing nuclear condensation with a reagent comprising a polypeptide comprising a polypeptide comprising a region at least 90% identical to the amino acid sequence of the GPCR-like protein encoded by the cDNA insert of the deposit comprising ATCC 98851, ATCC 98712, or ATCC 98852; and
(b) determining the suppression of induction of nuclear condensation in the sample compared to control containing staurosporine only.

21. A diagnostic process comprising analyzing for the presence of a nucleic acid of claims 3, 4, 5, 6, 7, 8, 9, 10, 11, 12 or 13 in a sample derived from a host.

22. A method for identifying compounds which regulate the activity of a GPCR-like protein of claim 1 comprising:
(a) incubating a sample comprising GPCR-like protein in a test medium containing said test compound and a reagent comprising a polypeptide comprising a region at least 90% identical to the amino acid sequence of insert of the deposit comprising ATCC 98851, ATCC 98712, or ATCC 98852;
(b) comparing the binding of said reagent to said protein in the sample in the presence and absence of said test compound; and
(c) relating the difference between the binding is step (b) to the test compound regulating the activity of the GPCR-like protein.

23. A method for identifying compounds which regulate the activity of a GPCR-like protein of claim 1 comprising:
(a) incubating a sample comprising GPCR-like protein in a test medium containing said test compound and a reagent comprising a polypeptide comprising a region at least 90% identical to the amino acid sequence of SEQ ID NOs: 4 or 6 under conditions effective for specific binding of said reagent to said GPCR-like protein; (b) comparing the binding of said reagent to said protein in the sample in the presence and absence of said test compound; and
(c) relating the difference between the binding is step (b) to the test compound regulating the activity of the GPCR-like protein.

24. A polypeptide with the amino acid sequence of SEQ ID NO: 2.

25. A polypeptide with the amino acid sequence of SEQ ID NO: 4.

26. A polypeptide with the amino acid sequence of SEQ ID NO: 6.
